(19) United States
(12) Patent Application Publication
(10) Pub. No.: US 2009/0054519 A1
(43) Pub. Date: Feb. 26, 2009

(54) DERIVATIVES OF MONOSACCHARIDES AS 5-LIPOXYGENASE INHIBITORS

(76) Inventors: Abhijit Ray, New Delhi (IN); Raj Kumar Shirumalla, New Delhi (IN); Kasim Mookhtiar, Mumbai (IN); Viswajanani J. Sattigeri, Gurgaon (IN); Venkata Palle, Pune (IN); Mohammad Salman, Princeton, NJ (US)

Correspondence Address: RANBAXY INC., 600 COLLEGE ROAD EAST, SUITE 2100, PRINCETON, NJ, 08540, US

(21) Appl. No.: 11/996,407

(22) PCT Filed: Jul. 20, 2006
(86) PCT No.: PCT/IB06/52497

§ 371 Date: Sep. 19, 2008

(30) Foreign Application Priority Data
Jul. 22, 2005 (IN) 1938/DEL/2005

(51) Int. Cl. A61K 31/36; A61P 43/00

(52) U.S. Cl. 514/466; 514/464

(57) ABSTRACT

The present invention relates to the use of derivatives of monosaccharides as 5-lipoxygenase inhibitor.

### FIELD OF INVENTION

The present invention relates to the use of derivatives of monosaccharides as 5-lipoxygenase inhibitor.

### BACKGROUND OF INVENTION

5-Lipoxygenase is a key enzyme that oxidizes arachidonic acid into biologically active leukotrienes, namely cysteinyl leukotrienes and leukotriene B4 (Clin. Exp. Allergy Rev., 1 196, 2001). Leukotrienes play important role in the pathophysiology of inflammatory/allergic diseases including bronchial asthma (Clin. Exp. Allergy Rev., 1, 264, 2001), allergic rhinitis (Clin. Exp. Allergy Rev. 1, 235, 2001), urticaria, atopic dermatitis (Clin. Exp. Allergy Rev. 1, 305, 2001), chronic obstructive pulmonary disease (Eur. Respir. J., 22, 926, 2003) etc. Incidence of allergic/inflammatory diseases are on the rise globally (Emerging Therapeutic Targets, 3, 229, 1999; Expert Opin. Investigational Drugs, 10, 1361, 2000).

A variety of stimuli namely antigen-antibody reaction, cold or hyperosmotic shock etc, that elevates intracellular calcium level, can evoke arachidonic acid release from cell membrane under the influence of cytosolic phospholipase A2. Arachidonic acid is transferred to nuclear membrane by 5-lipoxygenase binding protein (FLAP) and acted upon by 5-lipoxygenase enzyme to generate 5-hydroperoxyeicosatetraenoic acid (HPETE). HPETE is converted to LTA4 by 5-lipoxygenase. Depending upon cell type, LTA4 is converted to either cysteinyl leukotrienes and/or leukotriene B4 (Clin. Exp. Allergy Rev. 1, 196, 2001; Curr. Drug Targets—Inflammation &Allergy, 1, 23, 2002; Drug Safety, 26, 484, 2003).

Leukotrienes are generated by a variety of inflammatory cell types. Neutrophils and monocytes generate LTB4 whereas mast cells, basophils, eosinophils and bronchial epithelial cells produce cysteinyl leukotrienes. LTB4 acts as a chemoattractant for neutrophils through specific cell surface receptors. Cysteinyl leukotrienes, which include LTC4, LTD4 and LTE4, act on CysLT1 and CysLT2 receptors and increase bronchial smooth muscle contractility, promote mucosal secretion, increase vascular permeability and encourage eosinophils recruitment. (Am. J. Respir. Critic Care Med. 157, S210, 1998; Thorax 55, S32, 2000; Clin. Exp. Allergy Rev., 1, 196, 2001; Clin. Exp. Allergy Rev. 1, 220, 2001; Drug Safety, 26, 484, 2003).

There is evidence suggesting that cysteinyl leukotrienes can increase airway smooth muscle contractility in preclinical (Am. J. Respir Crit. Care Med. 157 S214, 1998) and clinical studies (Clin. Exp. Allergy Rev. 1, 220, 2001). Inhalation of leukotrienes also increase influx of inflammatory cells in the airway of animals (Clin. Exp. Allergy Rev. 1, 220, 2001) and humans (Am. J. Respir Crit. Care Med., 157, S210, 1998). In patients with asthma, urinary excretion of LTE4 correlates with exercise or cold air induced bronchoconstriction (Lancet, 1, 584, 1989) allergen induced early and late phase response (Clin. Exp Aller. 28, 1332, 1998; Am. J. Respir. Crit. Care Med 157, S210, 1998), as well as with reduction of FEV1 in patients with nocturnal asthma (Am. J. Respir. Crit. Care Med. 157, S233, 1998). Efficacy of leukotriene biosynthesis inhibitors and leukotriene receptor antagonists have been tested in numerous trials involving asthma patients (Clin. Exp. Aller. Review 1, 254, 2001; Drug Safety, 26, 483, 2003; NEJM, 340, 197, 1999; Am. J. Respir. Crit. Care Med. 157, S233, 1998).

Evidence is emerging that leukotrienes also contribute towards pathophysiology of COPD. Two major cell types, neutrophils and macrophages, that generate LTB4 and are modulated by the same in turn are believed to participate in the pathogenesis of COPD (Am. J. Respir. Crit. Care Med. 157, S210, 1998). Patients with COPD exhibit elevated sputum neutrophilia and LTB4 levels (Chest, 121, 197S, 2002). Elevated levels of LTB4 were shown to be present in the exhaled breath condensate of COPD patients (Thorax., 58, 585 2003) as well as in patients experiencing exacerbation of COPD (Thorax., 58, 294, 2003). Inhibitors of leukotriene biosynthesis as well as LTB4 receptor antagonists have shown to reduce airway reactivity, airway inflammation and airway neutrophilia in animals (J. Clin. Exp. Aller. 91, 917, 1992; J Pharmacol. Exp. Ther. 297, 458, 2000) as well as in human subjects (Thorax 51, 1178, 1996; Chest,. 122, 289, 2002). Cysteinyl leukotriene antagonists like Montelukast have shown protective effect in hypertonic saline induced bronchoconstriction in COPD patients (Eur. Respir. J. 22:926, 2003).

Similarly, evidence is emerging based on animal and human data that leukotriene pathway modulators can play role in arthritis (J. Pharmacol. Exp. Ther. 285, 946, 1998) allergic rhinitis and urticaria (Clin. Exp. Aller. Review, 1, 235, 2001)

Several leukotriene receptor antagonists, Montelukast, Zafirlukast, and Pranlukast, and one 5-lipoxygenase inhibitor, Zileuton, have been launched in the market. Both categories of molecules have shown efficacy in clinical trials of bronchial asthma. Inhibitors of 5-lipoxygenase exhibit greater potential to exhibit efficacy in COPD as well because of their inhibitory effect on LTB4 mediated processes. However, commercially available 5-lipoxygenase inhibitor is associated with poor pharmacokinetic properties and adverse events, like elevation of hepatic transaminase levels. This has prompted the search for novel inhibitors of 5-lipoxygenase with improved pharmacokinetic profiles and reduced adverse effects.

WO2004/071515 discloses preparation of monosaccharides derivatives for inhibition and prevention of cell adhesion and cell adhesion-mediated pathologies.

U.S. Pat. No. 6,329,344 discloses several derivatives of monosaccharides as cell adhesion inhibitors. U.S. Pat. No. 6,329,344 discloses classes of monosaccharides compounds useful for inhibitition and prevention of cell adhesion and cell adhesion mediated pathologies. U.S. Pat. No. 6,590,085 discloses further derivatives of monosaccharides similarly useful.

### SUMMARY OF THE INVENTION

According to one aspect a method for inhibiting the 5-Lipoxygenase enzyme is provided, comprising the administration to a patient in need of such treatment a therapeutically effective amount of a compound, having the structure of Formula I,

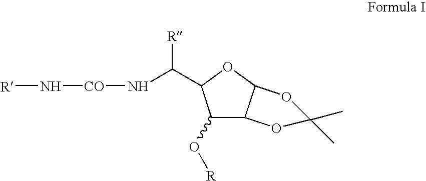

its pharmaceutically acceptable salts, esters, enantiomers, diastereomers, N-oxides, amides, prodrugs or metabolites
wherein,
R is C₁ to C₁₅ alkyl, alkene, alkyne (straight chain or branched), aryl, substituted aryl or alkylaryl;
R′ is SO₂C₆H₅, SO₂C₆H₄CH₃-p or SO₂C₆H₄—Cl, phenyl or substituted phenyl, represented as C₆H₄—R′″-p R′″ being Cl, NO₂, OCH₃, CH₃, CH₂COOH, CH₂COOCH₃, CH₂COLDVP, CH₂CODVP, CH₂COVP, wherein LDVP DVP and VP represent tetrapeptide (Leucyl-aspartyl-alyl-prolyl), tripeptide (aspartyl-valyl-prolyl) and dipeptide (valyl-prolyl) respectively;

### R″ is H or CH₃ and

[symbol] represents idofuranose, talofuranose, xylofuranose or ribofuranose configurations.

According to another aspect a method for inhibiting the 5-Lipoxygenase enzyme is provided, comprising the administration to a patient in need of such treatment a therapeutically effective amount of a compound, having the structure of Formula II

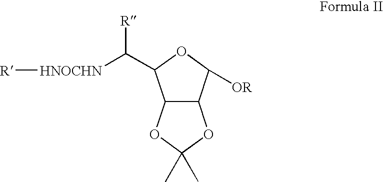

its pharmaceutically acceptable salts, esters, enantiomers, diastereomers, N-oxides, amides, prodrugs or metabolites, wherein
R is C₁ to C₁₅ alkyl, alkene, alkyne (straight chain or branched), aryl, substituted aryl or alkyl aryl;
R′ is SO₂C₆H₅, SO₂C₆H₅CH₃-p, SO₂C₆H₄Cl-p, phenyl or substituted phenyl, represented as C₆H₄—R′″-p, wherein R′″ being Cl, NO₂, OCH₃, CH₃, CH₂COOH, CH₂COOCH₃, CH₂COLDVP, CH₂CODVP, CH₂COVP, wherein LDVP, DVP and VP represent tetrapeptide (Leucyl-aspartyl-valyl-prolyl), tripeptide (aspartyl-valyl-prolyl) and dipeptide (valyl-prolyl), respectively; and

### R″ is H or CH₃.

According to a further aspect a method for inhibiting the 5 Lipoxygenase enzymeis provided, comprising the administration to a patient in need of such treatment a therapeutically effective amount of a compound, having the structure of Formula III,

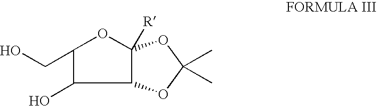

its pharmaceutically acceptable salts, esters, enantiomers, diastereomers, N-oxides, amides, prodrugs or metabolites
wherein
R′ is COLDVP, CODVP, COVP or CH₂NHCONHR″ wherein LDVP, DVP and VP represent tetrapeptide (Leucyl-aspartyl-valyl-prolyl), tripeptide (aspartyl-valyl-prolyl) and dipeptide (valyl-prolyl), respectively; R″ is C₆H₄R′″-p (R′″ is Cl, NO₂, OCH₃, CH₃, CH₂COOH, CH₂CO-LDVP, CH₂CO-DVP or CH₂CO—VP wherein LDVP, DVP and VP are the same as defined earlier).

According to yet another aspect, a method for inhibiting the 5-Lipoxygenase enzyme is provided, comprising the administration to a patient in need of such treatment a therapeutically effective amount of a compound, having the structure of Formula IV.

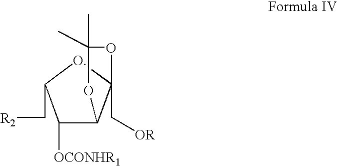

its pharmaceutically acceptable salts, esters, enantiomers, diastereomers, N-oxides, amides, prodrugs or metabolites, wherein
R is C₁ to C₁₅ alkyl, alkene, alkyne (straight chain or branched), aryl, substituted aryl or alkylaryl;
R₁ is phenyl, o-m- or p-chlorophenyl, tolyl, methoxyphenyl or nitrophenyl; and
R₂ is H, pyrrolidinyl, piperidinyl, morphilinyl or hexamethyleneimino or a radical of the Formula—NHR₃ wherein R₃ is C₁ to C₁₅ alkyl, alkene or alkyne (straight chain or branched) or a radical of the Formula

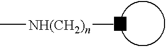

in which n is a whole number between 2 and 5 and

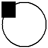

is a five, six or seven-membered, heterocyclic ring,
For example,

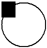

can be pyrrolidinyl, piperidinyl, morpholinyl or hexamethyleneimino moieties. It is another further aspect to provide novel methods for 5-lipoxygenase enzyme inhibition comprising the administration to a patient in need of such treatment a therapeutically effective amount of a compound or pharmaceutically acceptable salt of a compound of Formulae I, II, III and IV.

### DETAILED DESCRIPTION OF THE INVENTION

An illustrative list of particular compounds according to Formula I includes:
1,2-O-isopropylidene-3-O-dodecyl-5,6-dideoxy-5-N-{[4-(2-hydroxy-2-oxoethyl)phenyl]aminocarbonyl amino}-β-L-idofuranose (Compound No. 1),
1,2-O-isopropylidene-3-O-decyl-5,6-dideoxy-5-N-{[4-(2-hydroxy-2-oxoethyl)phenyl]aminocarbonylamino}-β-L-idofuranose (Compound No. 2),
1,2-O-isopropylidene-3-O-heptyl-5,6-dideoxy-5-N-{[4-(2-hydroxy-2-oxoethyl)phenyl]aminocarbonylamino}-β-L-idofuranose (Compound No. 3),
1,2-O-isopropylidene-3-O-butyl-5,6-dideoxy-5-N-{[4-(2-hydroxy-2-oxoethyl)phenyl]aminocarbonylamino}-β-L-idofuranose (Compound No. 4),
1,2-O-isopropylidene-3-O-dodecyl-5,6-dideoxy-5-N-{[4-chlorophenyl]aminocarbonylamino}-β-L-idofuranose (Compound No. 5),
1,2-O-isopropylidene-3-O-decyl-5,6-dideoxy-5-N-{[4-chlorophenyl]aminocarbonylamino}-β-L-idofuranose (Compound No. 6),
1,2-O-isopropylidene-3-O-heptyl-5,6-dideoxy-5-N-{[4-chlorophenyl]aminocarbonylamino}-β-L-idofuranose (Compound No. 7),
1,2-O-isopropylidene-3-O-butyl-5,6-dideoxy-5-N-{[4-chlorophenyl]aminocarbonylamino}-β-L-idofuranose (Compound No. 8),
1,2-O-isopropylidene-3-O-dodecyl-5,6-dideoxy-5-N-{[4-methoxyphenyl]amino carbonylamino}-β-L-idofuranose (Compound No. 9),
1,2-O-isopropylidene-3-O-decyl-5,6-dideoxy-5-N-{[4-methoxyphenyl]amino carbonylamino}-β-L-idofuranose (Compound No. 10),
1,2-O-isopropylidene-3-O-heptyl-5,6-dideoxy-5-N-{[4-methoxyphenyl]amino carbonylamino}-β-L-idofuranose (Compound No. 11),
1,2-O-isopropylidene-3-O-butyl-5,6-dideoxy-5-N-{[4-methoxyphenyl]aminocarbonylamino}-β-L-idofuranose (Compound No. 12),
1,2-O-isopropylidene-3-O-dodecyl-5,6-dideoxy-5-N-{[4-tolyl]aminocarbonylamino}-β-L-idofuranose (Compound No. 13),
1,2-O-isopropylidene-3-O-decyl-5,6-dideoxy-5-N-{[4-tolyl]aminocarbonylamino}-β-L-idofuranose (Compound No. 14),
1,2-O-isopropylidene-3-O-heptyl-5,6-dideoxy-5-N-{[4-tolyl]aminocarbonylamino}-β-L-idofuranose (Compound No. 15),
1,2-O-isopropylidene-3-O-butyl-5,6-dideoxy-5-N-{[4-tolyl]aminocarbonylamino}-β-L-idofuranose (Compound No. 16),
1,2-O-isopropylidene-3-O-dodecyl-5,6-dideoxy-5-N-{[4-(2-hydroxy-2-oxoethyl)phenyl]amino carbonyl amino}-β-L-talofuranose (Compound No. 17),
1,2-O-isopropylidene-3-O-decyl-5,6-dideoxy-5-N-{[4-(2-hydroxy-2-oxoethyl)phenyl aminocarbonylamino}-β-L-talofuranose (Compound No. 18),
1,2-O-isopropylidene-3-O-heptyl-5,6-dideoxy-5-N-{[4-(2-hydroxy-2-oxoethyl)phenyl]aminocarbonylamino}-β-L-talofuranose (Compound No. 19),
1,2-O-isopropylidene-3-O-butyl-5,6-dideoxy-5-N-{[4-(2-hydroxy-2-oxoethyl)phenyl]aminocarbonyl amino}-β-L-talofuranose (Compound No. 20),
1,2-O-isopropylidene-3-O-dodecyl-5,6-dideoxy-5-N-{[4-chlorophenyl]amino carbonylamino}-β-L-talofuranose (Compound No. 21),
1,2-O-isopropylidene-3-O-decyl-5,6-dideoxy-5-N-{[4-chlorophenyl]aminocarbonylamino}α,D-5-epiallofuranose (Compound No. 22),
1,2-O-isopropylidene-3-O-heptyl-5,6-dideoxy-5-N-{[4-chlorophenyl]aminocarbonylamino}-β-L-talofuranose (Compound No. 23),
1,2-O-isopropylidene-3-O-butyl-5,6-dideoxy-5-N-{[4-chlorophenyl]aminocarbonylamino}-β-L-talofuranose (Compound No. 24),
1,2-O-isopropylidene-3-O-dodecyl-5,6-dideoxy-5-N-{[4-methoxyphenyl]amino carbonylamino}-β-L-talofuranose (Compound No. 25),
1,2-O-isopropylidene-3-O-decyl-5,6-dideoxy-5-N-{[4-methoxyphenyl]aminocarbonylamino}-β-L-talofuranose (Compound No. 26),
1,2-O-isopropylidene-3-O-heptyl-5,6-dideoxy-5-N-{[4-methoxyphenyl]aminocarbonyl amino}-β-L-talofuranose (Compound No. 27),
1,2-O-isopropylidene-3-O-butyl-5,6-dideoxy-5-N-{[4-methoxyphenyl]amino carbonylamino}-β-L-talofuranose (Compound No. 28),
1,2-O-isopropylidene-3-O-dodecyl-5,6-dideoxy-5-N-{[4-tolyl]aminocarbonylamino}-β-L-talofuranose (Compound No. 29),
1,2-O-isopropylidene-3-O-decyl-5,6-dideoxy-5-N-{[4-tolyl]aminocarbonylamino}-β-L-talofuranose (Compound No. 30),
1,2-O-isopropylidene-3-O-heptyl-5,6-dideoxy-5-N-{[4-tolyl]aminocarbonylamino}-β-L-talofuranose (Compound No. 31),
1,2-O-isopropylidene-3-O-butyl-5,6-dideoxy-5-N-{[4-tolyl]aminocarbonylamino}-β-L-talofuranose (Compound No. 32),
1,2-O-isopropylidene-3-O-dodecyl-5-deoxy-5-N-{[4-(2-hydroxy-2-oxoethyl)phenyl]aminocarbonylamino}-(α,D-xylofuranose (Compound No. 33),
1,2-O-isopropylidene-3-O-decyl-5-deoxy-5-N-{[4-(2-hydroxy-2-oxoethyl)phenyl]aminocarbonylamino}-(α,D-xylofuranose (Compound No. 34),
1,2-O-isopropylidene-3-O-hexyl-5-deoxy-5-N-{[4-(2-hydroxy-2-oxoethyl)phenyl]aminocarbonylamino}-(α,D-xylofuranose (Compound No. 35),
1,2-O-isopropylidene-3-O-butyl-5-deoxy-5-N-{[4-(2-hydroxy-2-oxoethyl)phenyl]aminocarbonylamino}-(α,D-xylofuranose (Compound No. 36),
1,2-O-isopropylidene-3-O-methyl-5-deoxy-5-N-{[4-(2-hydroxy-2-oxoethyl)phenyl]aminocarbonylamino}-(α,D-xylofuranose (Compound No. 37),
1,2-O-isopropylidene-3-O-dodecyl-5-deoxy-5-N-{[4-chlorophenyl]amino carbonylamino}α,D-xylofuranose (Compound No. 38),
1,2-O-isopropylidene-3-O-decyl-5-deoxy-5-N-{[4-chlorophenyl]amino carbonylamino}α,D-xylofuranose (Compound No. 39),
1,2-O-isopropylidene-3-O-hexyl-5-deoxy-5-N-{[4-chlorophenyl]amino carbonylamino}α,D-xylofuranose (Compound No. 40),
1,2-O-isopropylidene-3-O-butyl-5-deoxy-5-N-{[4-chlorophenyl]amino carbonylamino}α,D-xylofuranose (Compound No. 41),
1,2-O-isopropylidene-3-O-methyl-5-deoxy-5-N-{[4-chlorophenyl]amino carbonylamino}α,D-xylofuranose (Compound No. 42),
1,2-O-isopropylidene-3-O-dodecyl-5-deoxy-5-N-{[4-methoxyphenyl]amino carbonylamino}α,D-xylofuranose (Compound No. 43),
1,2-O-isopropylidene-3-O-decyl-5-deoxy-5-N-{[4-methoxyphenyl]aminocarbonylamino}α,D-xylofuranose (Compound No. 44),
1,2-O-isopropylidene-3-O-hexyl-5-deoxy-5-N-{[4-methoxyphenyl]aminocarbonylamino}α,D-xylofuranose (Compound No. 45),
1,2-O-isopropylidene-3-O-butyl-5-deoxy-5-N-{[4-methoxyphenyl]aminocarbonylamino}α,D-xylofuranose (Compound No. 46),
1,2-O-isopropylidene-3-O-methyl-5-deoxy-5-N-{[4-methoxyphenyl]aminocarbonylamino}α,D-xylofuranose (Compound No. 47),
1,2-O-isopropylidene-3-O-dodecyl-5-deoxy-5-N-{[4-tolyl]aminocarbonylamino}β, D-xylofuranose (Compound No. 48),
1,2-O-isopropylidene-3-O-decyl-5-deoxy-5-N-{[4-tolyl]aminocarbonylamino}α,D-xylofuranose (Compound No. 49),
1,2-O-isopropylidene-3-O-hexyl-5-deoxy-5-N-{[4-tolyl]aminocarbonylamino}α,D-xylofuranose (Compound No. 50),
1,2-O-isopropylidene-3-O-butyl-5-deoxy-5-N-{[4-tolyl]aminocarbonylamino}α,D-xylofuranose (Compound No. 51),
1,2-O-isopropylidene-3-O-methyl-5-deoxy-5-N-{[4-tolyl]aminocarbonylamino}α,D-xylofuranose (Compound No. 52),
1,2-O-isopropylidene-3-O-dodecyl-5-deoxy-5-N-{[aminocarbonylamino]phenyl acetyl-L-Leucyl-α,L-Aspartyl-L-Valyl-L-Proline}-α,D-xylofuranose (Compound No. 53),
1,2-O-isopropylidene-3-O-dodecyl-5-deoxy-5-N-{[aminocarbonylamino]phenylacetyl-α,L-Aspartyl-L-Valyl-L-Proline}-α,D-xylofuranose (Compound No. 54),
1,2-O-isopropylidene-3-O-dodecyl-5-deoxy-5-N-{[aminocarbonylamino]phenylacetyl-alyl-L-Proline}-α,D-xylofuranose (Compound No. 55)
1,2-O-isopropylidene-3-O-dodecyl-5-deoxy-5-N-{[4-(2-hydroxy-2-oxoethyl)phenyl]aminocarbonylamino}-α,D-ribofuranose (Compound No. 56),
1,2-O-isopropylidene-3-O-decyl-5-deoxy-5-N-{[4-(2-hydroxy-2-oxoethyl)phenyl]aminocarbonylamino}-α,D-ribofuranose (Compound No. 57),
1,2-O-isopropylidene-3-O-heptyl-5-deoxy-5-N-{[4-(2-hydroxy-2-oxoethyl)phenyl]aminocarbonylamino}-α,D-ribofuranose (Compound No. 58),
1,2-O-isopropylidene-3-O-butyl-5-deoxy-5-N-{[4-(2-hydroxy-2-oxoethyl)phenyl]aminocarbonylamino}-α,D-ribofuranose (Compound No. 59),
1,2-O-isopropylidene-3-O-methyl-5-deoxy-5-N-{[4-(2-hydroxy-2-oxoethyl)phenyl]aminocarbonylamino}-α,D-ribofuranose (Compound No. 60),
1,2-O-isopropylidene-3-O-dodecyl-5-deoxy-5-N-{[4-chlorophenyl]aminocarbonylamino}α,D-ribofuranose (Compound No. 61),
1,2-O-isopropylidene-3-O-decyl-5-deoxy-5-N-{[4-chlorophenyl]aminocarbonylamino}α,D-ribofuranose (Compound No. 62),
1,2-O-isopropylidene-3-O-heptyl-5-deoxy-5-N-{[4-chlorophenyl]aminocarbonylamino}1α,D-ribofuranose (Compound No. 63),
1,2-O-isopropylidene-3-O-butyl-5-deoxy-5-N-{[4-chlorophenyl]aminocarbonylamino}α,D-ribofuranose (Compound No. 64),
1,2-O-isopropylidene-3-O-methyl-5-deoxy-5-N-{[4-chlorophenyl]aminocarbonylamino}α,D-ribofuranose (Compound No. 65),
1,2-O-isopropylidene-3-O-dodecyl-5-deoxy-5-N-{[4-methoxyphenyl]aminocarbonylamino}α,D-ribofuranose (Compound No. 66),
1,2-O-isopropylidene-3-O-decyl-5-deoxy-5-N-{[4-methoxyphenyl]aminocarbonylamino}α,D-ribofuranose (Compound No. 67),
1,2-O-isopropylidene-3-O-heptyl-5-deoxy-5-N-{[4-methoxyphenyl]aminocarbonylamino}1α,D-ribofuranose (Compound No. 68),
1,2-O-isopropylidene-3-O-butyl-5-deoxy-5-N-{[4-methoxyphenyl]aminocarbonylamino}α,D-ribofuranose (Compound No. 69),
1,2-O-isopropylidene-3-O-methyl-5-deoxy-5-N-{[4-methoxyphenyl]aminocarbonylamino}α,D-ribofuranose (Compound No. 70),
1,2-O-isopropylidene-3-O-dodecyl-5-deoxy-5-N-{[4-tolyl]aminocarbonylamino}α, D-ribofuranose (Compound No. 71),
1,2-O-isopropylidene-3-O-decyl-5-deoxy-5-N-{[4-tolyl]aminocarbonylamino}α, D-ribofuranose (Compound No. 72),
1,2-O-isopropylidene-3-O-heptyl-5-deoxy-5-N-{[4-tolyl]aminocarbonylamino}α, D-ribofuranose (Compound No. 73),
1,2-O-isopropylidene-3-O-butyl-5-deoxy-5-N-{[4-tolyl]aminocarbonylamino}α, D-ribofuranose (Compound No. 74),
1,2-O-isopropylidene-3-O-methyl-5-deoxy-5-N-{[4-tolyl]aminocarbonylamino}β, D-ribofuranose (Compound No. 75),
1,2-O-isopropylidene-3-O-dodecyl-5-deoxy-5-N-{[aminocarbonylaminophenyl]acetyl-L-Leucyl-α,L-Aspartyl-L-Valyl-L-Proline}-α,D-ribofuranose (Compound No. 76),
1,2-O-isopropylidene-3-O-dodecyl-5-deoxy-5-N-{[aminocarbonylaminophenyl]acetyl-α,L-Aspartyl-L-Valyl-L-Proline}-α,D-ribofuranose (Compound No. 77), and
1,2-O-isopropylidene-3-O-dodecyl-5-deoxy-5-N-{[aminocarbonylaminophenyl]acetyl-L-Valyl-L-Proline}-α,D-ribofuranose (Compound No. 78).
   An illustrative list of particular compounds according to Formula II includes:
2,3-O-isopropylidene-1-O-butyl-5,6-dideoxy-5-N-{[4-(2-hydroxy-2-oxoethyl)phenyl]aminocarbonyl amino}-β,L-gulofuranoside (Compound No. 79),
2,3-O-isopropylidene-1-O-hexyl-5,6-dideoxy-5-N-{[4-(2-hydroxy-2-oxoethyl)phenyl]aminocarbonyl amino}-β,L-gulofuranoside (Compound No. 80),
2,3-O-isopropylidene-1-O-heptyl-5,6-dideoxy-5-N-{[4-(2-hydroxy-2-oxoethyl)phenyl]aminocarbonyl amino}-β,L-gulofuranoside (Compound No. 81),
2,3-O-isopropylidene-1-O-decyl-5,6-dideoxy-5-N-{[4-(2-hydroxy-2-oxoethyl)phenyl]aminocarbonyl amino}-β,L-gulofuranoside (Compound No. 82),
2,3-O-isopropylidene-1-O-dodecyl-5,6-dideoxy-5-N-{[4-(2-hydroxy-2-oxoethyl)phenyl]aminocarbonylamino}-β,L-gulofuranoside (Compound No. 83),
Tris salt of 2,3-O-isopropylidene-1-O-dodecyl-5,6-dideoxy-5-N-{[4-(2-hydroxy-2-oxoethyl)phenyl]aminocarbonylamino}-β,L-gulofuranoside (Compound No. 84)
Sodium salt of 2,3-O-isopropylidene-1-O-dodecyl-5,6-dideoxy-5-N-{[4-(2-hydroxy-2-oxoethyl)phenyl]aminocarbonylamino}-β,L-gulofuranoside (Compound No. 85)
2,3-O-isopropylidene-1-O-butyl-5,6-dideoxy-5-N-{[4-chlorophenyl]aminocarbonylamino}-β,L-gulofuranoside (Compound No. 86),
2,3-O-isopropylidene-1-O-hexyl-5,6-dideoxy-5-N-{[4-chlorophenyl]aminocarbonylamino}-β,L-gulofuranoside (Compound No. 87),
2,3-O-isopropylidene-1-O-heptyl-5,6-dideoxy-5-N-{[4-chlorophenyl]aminocarbonylamino}-β,L-gulofuranoside (Compound No. 88),
2,3-O-isopropylidene-1-O-decyl-5,6-dideoxy-5-N-{[4-chlorophenyl]aminocarbonylamino}-β,L-gulofuranoside (Compound No. 89),
2,3-O-isopropylidene-1-O-dodecyl-5,6-dideoxy-5-N-{[4-chlorophenyl]aminocarbonylamino}-β,L-gulofuranoside (Compound No. 90),
2,3-O-isopropylidene-1-O-butyl-5,6-dideoxy-5-N-{[4-methoxyphenyl]aminocarbonylamino}-β,L-gulofuranoside (Compound No. 91),
2,3-O-isopropylidene-1-O-hexyl-5,6-dideoxy-5-N-{[4-methoxyphenyl]aminocarbonylamino}-β,L-gulofuranoside (Compound No. 92),
2,3-O-isopropylidene-1-O-heptyl-5,6-dideoxy-5-N-{[4-methoxyphenyl]aminocarbonylamino}-β,L-gulofuranoside (Compound No. 93),
2,3-O-isopropylidene-1-O-decyl-5,6-dideoxy-5-N-{[4-methoxyphenyl]aminocarbonylamino}-β,L-gulofuranoside (Compound No. 94),
2,3-O-isopropylidene-1-O-dodecyl-5,6-dideoxy-5-N-{[4-methoxyphenyl]amino carbonylamino}-β,L-gulofuranoside (Compound No. 95),
2,3-O-isopropylidene-1-O-butyl-5,6-dideoxy-5-N-{[4-tolyl]aminocarbonylamino}-α,L-gulofuranoside (Compound No. 96),
2,3-O-isopropylidene-1-O-hexyl-5,6-dideoxy-5-N-{[4-tolyl]aminocarbonylamino}-β,L-gulofuranoside (Compound No. 97),
2,3-O-isopropylidene-1-O-heptyl-5,6-dideoxy-5-N-{[4-tolyl]aminocarbonylamino}-β,L-gulofuranoside (Compound No. 98),
2,3-O-isopropylidene-1-O-decyl-5,6-dideoxy-5-N-{[4-tolyl]aminocarbonylamino}-β,L-gulofuranoside (Compound No. 99),
2,3-O-isopropylidene-1-O-dodecyl-5,6-dideoxy-5-N-{[4-tolyl]aminocarbonylamino}-β,L-gulofuranoside (Compound No. 100),
2,3-O-isopropylidene-1-O-methyl-5-deoxy-5-N-{[4-(2-hydroxy-2-oxoethyl)phenyl]aminocarbonylamino}-α,D-lyxofuranoside (Compound No. 101),
2,3-O-isopropylidene-1-O-butyl-5-deoxy-5-N-{[4-(2-hydroxy-2-oxoethyl)phenyl]aminocarbonylamino}-α,D-lyxofuranoside (Compound No. 102),
2,3-O-isopropylidene-1-O-heptyl-5-deoxy-5-N-{[4-(2-hydroxy-2-oxoethyl)phenyl]aminocarbonylamino}-α,D-lyxofuranoside (Compound No. 103),
2,3-O-isopropylidene-1-O-decyl-5-deoxy-5-N-{[4-(2-hydroxy-2-oxoethyl)phenyl]aminocarbonylamino}-α,D-lyxofuranose (Compound No. 104),
2,3-O-isopropylidene-1-O-dodecyl-5-deoxy-5-N-{[4-(2-hydroxy-2-oxoethyl)phenyl]aminocarbonyl amino}-α,D-lyxofuranoside (Compound No. 105),
2,3-O-isopropylidene-1-O-methyl-5-deoxy-5-N-{[4-chlorophenyl]aminocarbonylamino}α,D-lyxofuranoside (Compound No. 106),
2,3-O-isopropylidene-1-O-butyl-5-deoxy-5-N-{[4-chlorophenyl]aminocarbonylamino}α,D-lyxofuranoside (Compound No. 107),
2,3-O-isopropylidene-1-O-heptyl-5-deoxy-5-N-{[4-chlorophenyl]aminocarbonylamino}α,D-lyxofuranoside (Compound No. 108),
2,3-O-isopropylidene-1-O-decyl-5-deoxy-5-N-{[4-chlorophenyl]aminocarbonylamino}α,D-lyxofuranoside (Compound No. 109),
2,3-O-isopropylidene-1-O-dodecyl-5-deoxy-5-N-{[4-chlorophenyl]aminocarbonylamino}α,D-lyxofuranoside (Compound No. 110),
2,3-O-isopropylidene-1-O-methyl-5-deoxy-5-N-{[4-methoxyphenyl]aminocarbonylamino}α,D-lyxofuranoside (Compound No. 111),
2,3-O-isopropylidene-1-O-butyl-5-deoxy-5-N-{[4-methoxyphenyl]aminocarbonylamino}α,D-lyxofuranoside (Compound No. 112),
2,3-O-isopropylidene-1-O-heptyl-5-deoxy-5-N-{[4-methoxyphenyl]aminocarbonyl amino}α,D-lyxofuranoside (Compound No. 113),
2,3-O-isopropylidene-1-O-decyl-5-deoxy-5-N-{[4-methoxyphenyl]aminocarbonylamino}α,D-lyxofuranoside (Compound No. 114),
2,3-O-isopropylidene-1-O-dodecyl-5-deoxy-5-N-{[4-methoxyphenyl]amino carbonylamino}αD-lyxofuranoside (Compound No. 115),
2,3-O-isopropylidene-1-O-methyl-5-deoxy-5-N-{[4-tolyl]aminocarbonylamino}α, D-lyxofuranoside (Compound No. 116),
2,3-O-isopropylidene-1-O-butyl-5-deoxy-5-N-{[4-tolyl]aminocarbonylamino}α, D-lyxofuranoside (Compound No. 117),
2,3-O-isopropylidene-1-O-heptyl-5-deoxy-5-N-{[4-tolyl]aminocarbonylamino}α, D-lyxofuranoside (Compound No. 118),
2,3-O-isopropylidene-1-O-decyl-5-deoxy-5-N-{[4-tolyl]aminocarbonylamino}α, D-lyxofuranoside (Compound No. 119),
2,3-O-isopropylidene-1-O-dodecyl-5-deoxy-5-N-{[4-tolyl]aminocarbonylamino}α, D-lyxofuranoside (Compound No. 120),
2,3-O-isopropylidene-1-O-dodecyl-5-deoxy-5-N-{[aminocarbonylamino]phenylacetyl-L-Leucyl-α,L-Aspartyl-L-Valyl-L-Proline}-α,D-lyxofuranoside (Compound No. 121),
2,3-O-isopropylidene-1-O-dodecyl-5-deoxy-5-N-{[aminocarbonylamino]phenyl acetyl-α,L-Aspartyl-L-Valyl-L-Proline}-α,D-lyxofuranoside (Compound No. 122), and
2,3-O-isopropylidene-1-O-dodecyl-5-deoxy-5-N-{[aminocarbonylamino]phenyl acetyl-L-Valyl-L-Proline}-α,D-lyxofuranoside (Compound No. 123).
   An illustrative list of particular compounds according to Formula III includes:
2,3;4,6-Di-O-isopropylidene-1-carbonyl-L-Leucyl-α,L-Aspartyl-L-Valyl-L-Proline-α, L-xylo-2-hexulofuranosonic acid (Compound No. 124),
2,3;4,6-Di-O-isopropylidene-1-carbonyl-α,L-Aspartyl-L-Valyl-L-Proline-α,L-xylo-2-hexulofuranosonic acid (Compound No. 125),
2,3-O-isopropylidene-1-carbonyl-L-Leucyl-α,L-Aspartyl-L-Valyl-L-Proline-α,L-xylo-2-hexulofuranosonic acid (Compound No. 126),
2,3-O-isopropylidene-1-carbonyl-α,L-Aspartyl-L-Valyl-L-Proline-α,L-xylo-2-hexulofuranosonic acid (Compound No. 127),
2,3-O-isopropylidene-1-carbonyl-L-Valyl-L-Proline-α,L-xylo-2-hexulofuranosonic acid (Compound No. 128),
2,3;4,6-Di-O-isopropylidene-1-deoxy-1-N-{[4-chlorophenyl]aminocarbonylamino}-α,L-xylo-2-hexulofuranose (Compound No. 129),
2,3;4,6-Di-O-isopropylidene-1-deoxy-1-N-{[4-methoxyphenyl]aminocarbonylamino}-α,L-xylo-2-hexulofuranose (Compound No. 130),
2,3;4,6-Di-O-isopropylidene-1-deoxy-1-N-{[4-tolyl]aminocarbonylamino}-α,L-xylo-2-hexulofuranose (Compound No. 131),
2,3-O-isopropylidene-1-deoxy-1-N-{[4-(2-hydroxy-2oxoethyl))phenylaminocarbonylamino}-α,L-xylo-2-hexulofuranose (Compound No. 132),
2,3-O-isopropylidene-1-deoxy-1-N-{[4-chlorophenyl]aminocarbonylamino}-α,L-xylo-2-hexulofuranose (Compound No. 133),
2,3-O-isopropylidene-1-deoxy-1-N-{[4-methoxyphenyl]aminocarbonylamino}-α,L-xylo-2-hexulofuranose (Compound No. 134),
2,3-O-isopropylidene-1-deoxy-1-N-{[4-tolyl]aminocarbonylamino}-α,L-xylo-2-hexulofuranose (Compound No. 135),
2,3-O-isopropylidene-1 deoxy-1-N-{[aminocarbonylaminophenylacetyl-L-Leucyl-α,L-Aspartyl-L-Valyl-L-Proline)}-α,L-xylo-2-hexulofuranose (Compound No. 136),
2,3-O-isopropylidene-1-deoxy-1-N-{[aminocarbonylaminophenylacetyl-α,L-Aspartyl-L-Valyl-L-Proline)}-α,L-xylo-2-hexulofuranose (Compound No. 137),
2,3-O-isopropylidene-1 deoxy-1-N-{[aminocarbonylaminophenylacetyl-L-Valyl-L-Proline)}-α,L-xylo-2-hexulofuranose (Compound No. 138).
   An illustrative list of particular compounds according to Formula IV include:
2,3-O-Isopropylidene-1-O-decyl-4-O-(methylaminocarbonyl)-6-deoxy-6-pyrrolidin-1-yl-α-L-xylo-2-hexulofuranose (Compound No. 139),
2,3-O-Isopropylidene-1-O-dodecyl-4-O-(methylaminocarbonyl)-6-deoxy-6-pyrrolidin-1-yl-α-L-xylo-2-hexulofuranose (Compound No. 140),
2,3-O-Isopropylidene-1-O-dodecyl-4-O-(methylaminocarbonyl)-6-deoxy-6-pyrrolidin-1-yl-α-L-xylo-2-hexulofuranose (Compound No. 141),
2,3-O-Isopropylidene-1-O-dodecyl-4-O-(4-chlorophenylaminocarbonyl)-6-deoxy-6-pyrrolidin-1-yl-α-L-xylo-2-hexulofuranose (Compound No. 142),
2,3-O-Isopropylidene-1-O-dodecyl-4-O-(4-tolylaminocarbonyl)-6-deoxy-6-pyrrolidin-1-yl-α-L-xylo-2-hexulofuranose (Compound No. 143),
2,3-O-Isopropylidene-1-O-dodecyl-4-O-(4-methoxyphenylaminocarbonyl)-6-deoxy-6-pyrrolidin-1-yl-α-L-xylo-2-hexulofuranose (Compound No. 144)
2,3-O-Isopropylidene-1-O-dodecyl-4-O-(4-nitrophenylaminocarbonyl)-6-deoxy-6-pyrrolidin-1-yl-α-L-xylo-2-hexulofuranose (Compound No. 145),
2,3-O-Isopropylidene-1-O-decyl-4-O-(phenylaminocarbonyl)-6-deoxy-6-pyrrolidin-1-yl-α-L-xylo-2-hexulofuranose (Compound No. 146),
2,3-O-Isopropylidene-1-O-decyl-4-O-(4-chlorophenylaminocarbonyl)-6-deoxy-6-pyrrolidin-1-yl-α-L-xylo-2-hexulofuranose (Compound No. 147),
2,3-O-Isopropylidene-1-O-decyl-4-O-(4-tolylaminocarbonyl)-6-deoxy-6-pyrrolidin-1-yl-α-L-xylo-2-hexulofuranose (Compound No. 148),
2,3-O-Isopropylidene-1-O-decyl-4-O-(4-methoxyphenylaminocarbonyl)-6-deoxy-6-pyrrolidin-1-yl-α-L-xylo-2-hexulofuranose (Compound No. 149),
2,3-O-Isopropylidene-1-O-decyl-4-O-(4-nitrophenylaminocarbonyl)-6-deoxy-6-pyrrolidin-1-yl-α-L-xylo-2-hexulofuranose (Compound No. 150),
2,3-O-Isopropylidene-1-O-heptyl-4-O-(phenylaminocarbonyl)-6-deoxy-6-pyrrolidin-1-yl-α-L-xylo-2-hexulofuranose (Compound No. 151),
2,3-O-Isopropylidene-1-O-heptyl-4-O-(4-chlorophenylaminocarbonyl)-6-deoxy-6-pyrrolidin-1-yl-α-L-xylo-2-hexulofuranose (Compound No. 152),
2,3-O-Isopropylidene-1-O-heptyl-4-O-(4-tolylaminocarbonyl)-6-deoxy-6-pyrrolidin-1-yl-α-L-xylo-2-hexulofuranose (Compound No. 153),
2,3-O-Isopropylidene-1-O-heptyl-4-O-(4-methoxyphenylaminocarbonyl)-6-deoxy-6-pyrrolidin-1-yl-α-L-xylo-2-hexulofuranose (Compound No. 154),
2,3-O-Isopropylidene-1-O-heptyl-4-O-(4-nitrophenylaminocarbonyl)-6-deoxy-6-pyrrolidin-1-yl-α-L-xylo-2-hexulofuranose (Compound No. 155),
2,3-O-Isopropylidene-1-O-dodecyl-4-O-(phenylaminocarbonyl)-6-deoxy-6-morpholin-4-yl-α-L-xylo-2-hexulofuranose (Compound No. 156),
2,3-O-Isopropylidene-1-O-dodecyl-4-O-(4-chlorophenylaminocarbonyl)-6-deoxy-6-morpholin-4-yl-α-L-xylo-2-hexulofuranose (Compound No. 157),
2,3-O-Isopropylidene-1-O-dodecyl-4-O-(4-tolylaminocarbonyl)-6-deoxy-6-morpholin-4-yl-α-L-xylo-2-hexulofuranose (Compound No. 158),
2,3-O-Isopropylidene-1-O-dodecyl-4-O-(4-methoxyphenylaminocarbonyl)-6-deoxy-6-morpholin-4-yl-α-L-xylo-2-hexulofuranose (Compound No. 159),
2,3-O-Isopropylidene-1-O-dodecyl-4-O-(4-nitrophenylaminocarbonyl)-6-deoxy-6-morpholin-4-yl-α-L-xylo-2-hexulofuranose (Compound No. 160),
2,3-O-Isopropylidene-1-O-decyl-4-O-(phenylaminocarbonyl)-6-deoxy-6-morpholin-4-yl-α-L-xylo-2-hexulofuranose (Compound No. 161),
2,3-O-Isopropylidene-1-O-decyl-4-O-(4-chlorophenylaminocarbonyl)-6-deoxy-6-morpholin-4-yl-α-L-xylo-2-hexulofuranose (Compound No. 162),
2,3-O-Isopropylidene-1-O-decyl-4-O-(4-tolylaminocarbonyl)-6-deoxy-6-morpholin-4-yl-α-L-xylo-2-hexulofuranose (Compound No. 163),
2,3-O-Isopropylidene-1-O-decyl-4-O-(4-methoxyphenylaminocarbonyl)-6-deoxy-6-morpholin-4-yl-α-L-xylo-2-hexulofuranose (Compound No. 164),
2,3-O-Isopropylidene-1-O-decyl-4-O-(4-nitrophenylaminocarbonyl)-6-deoxy-6-morpholin-4-yl-α-L-xylo-2-hexulofuranose (Compound No. 165),
2,3-O-Isopropylidene-1-O-heptyl-4-O-(phenylaminocarbonyl)-6-deoxy-6-morpholin-4-yl-α-L-xylo-2-hexulofuranose (Compound No. 166),
2,3-O-Isopropylidene-1-O-heptyl-4-O-(4-chlorophenylaminocarbonyl)-6-deoxy-6-morpholin-4-yl-α-L-xylo-2-hexulofuranose (Compound No. 167),
2,3-O-Isopropylidene-1-O-heptyl-4-O-(4-tolylaminocarbonyl)-6-deoxy-6-morpholin-4-yl-α-L-xylo-2-hexulofuranose (Compound No. 168),
2,3-O-Isopropylidene-1-O-heptyl-4-O-(4-methoxyphenylaminocarbonyl)-6-deoxy-6-morpholin-4-yl-α-L-xylo-2-hexulofuranose (Compound No. 169),
2,3-O-Isopropylidene-1-O-heptyl-4-O-(4-nitrophenylaminocarbonyl)-6-deoxy-6-morpholin-4-yl-α-L-xylo-2-hexulofuranose (Compound No. 170),
2,3-O-Isopropylidene-1-O-dodecyl-4-O-(phenylaminocarbonyl)-6-deoxy-6-piperidin-1-yl-α-L-xylo-2-hexulofuranose (Compound No. 171),
2,3-O-Isopropylidene-1-O-dodecyl-4-O-(4-chlorophenylaminocarbonyl)-6-deoxy-6-piperidin-1-yl-α-L-xylo-2-hexulofuranose (Compound No. 172),
2,3-O-Isopropylidene-1-O-dodecyl-4-O-(4-tolylaminocarbonyl)-6-deoxy-6-piperidin-1-yl-α-L-xylo-2-hexulofuranose (Compound No. 173),
2,3-O-Isopropylidene-1-O-dodecyl-4-O-(4-methoxyphenylaminocarbonyl)-6-deoxy-6-piperidin-1-yl-α-L-xylo-2-hexulofuranose (Compound No. 174),
2,3-O-Isopropylidene-1-O-dodecyl-4-O-(4-nitrophenylaminocarbonyl)-6-deoxy-6-piperidin-1-yl-α-L-xylo-2-hexulofuranose (Compound No. 175),
2,3-O-Isopropylidene-1-O-decyl-4-O-(phenylaminocarbonyl)-6-deoxy-6-piperidin-1-yl-α-L-xylo-2-hexulofuranose (Compound No. 176),
2,3-O-Isopropylidene-1-O-decyl-4-O-(4-chlorophenylaminocarbonyl)-6-deoxy-6-piperidin-1-yl-α-L-xylo-2-hexulofuranose (Compound No. 177),
2,3-O-Isopropylidene-1-O-decyl-4-O-(4-tolylaminocarbonyl)-6-deoxy-6-piperidin-1-yl-α-L-xylo-2-hexulofuranose (Compound No. 178),
2,3-O-Isopropylidene-1-O-decyl-4-O-(4-methoxyphenylaminocarbonyl)-6-deoxy-6-piperidin-1-yl-α-L-xylo-2-hexulofuranose (Compound No. 179),
2,3-O-Isopropylidene-1-O-decyl-4-O-(4-nitrophenylaminocarbonyl)-6-deoxy-6-piperidin-1-yl-α-L-xylo-2-hexulofuranose (Compound No. 180),
2,3-O-Isopropylidene-1-O-heptyl-4-O-(phenylaminocarbonyl)-6-deoxy-6-piperidin-1-yl-α-L-xylo-2-hexulofuranose (Compound No. 181),
2,3-O-Isopropylidene-1-O-heptyl-4-O-(4-chlorophenylaminocarbonyl)-6-deoxy-6-piperidin-1-yl-α-L-xylo-2-hexulofuranose (Compound No. 182),
2,3-O-Isopropylidene-1-O-heptyl-4-O-(4-tolylaminocarbonyl)-6-deoxy-6-piperidin-1-yl-α-L-xylo-2-hexulofuranose (Compound No. 183),
2,3-O-Isopropylidene-1-O-heptyl-4-O-(4-methoxyphenylaminocarbonyl)-6-deoxy-6-piperidin-1-yl-α-L-xylo-2-hexulofuranose (Compound No. 184),
2,3-O-Isopropylidene-1-O-heptyl-4-O-(4-nitrophenylaminocarbonyl)-6-deoxy-6-piperidin-1-yl-α-L-xylo-2-hexulofuranose (Compound No. 185),
2,3-O-Isopropylidene-1-O-dodecyl-4-O-(phenylaminocarbonyl)-6-deoxy-6-azepan-1-yl-α-L-xylo-2-hexulofuranose (Compound No. 186),
2,3-O-Isopropylidene-1-O-dodecyl-4-O-(4-chlorophenylaminocarbonyl)-6-deoxy-6-azepan-1-yl-α-L-xylo-2-hexulofuranose (Compound No. 187),
2,3-O-Isopropylidene-1-O-dodecyl-4-O-(4-tolylaminocarbonyl)-6-deoxy-6-azepan-1-yl-α-L-xylo-2-hexulofuranose (Compound No. 188),
2,3-O-Isopropylidene-1-O-dodecyl-4-O-(4-methoxyphenylaminocarbonyl)-6-deoxy-6-azepan-1-yl-α-L-xylo-2-hexulofuranose (Compound No. 189),
2,3-O-Isopropylidene-1-O-dodecyl-4-O-(4-nitrophenylaminocarbonyl)-6-deoxy-6-azepan-1-yl-α-L-xylo-2-hexulofuranose (Compound No. 190),
2,3-O-Isopropylidene-1-O-decyl-4-O-(phenylaminocarbonyl)-6-deoxy-6-azepan-1-yl-α-L-xylo-2-hexulofuranose (Compound No. 191),
2,3-O-Isopropylidene-1-O-decyl-4-O-(4-chlorophenylaminocarbonyl)-6-deoxy-6-azepan-1-yl-α-L-xylo-2-hexulofuranose (Compound No. 192),
2,3-O-Isopropylidene-1-O-decyl-4-O-(4-tolylaminocarbonyl)-6-deoxy-6-azepan-1-yl-α-L-xylo-2-hexulofuranose (Compound No. 193),
2,3-O-Isopropylidene-1-O-decyl-4-O-(4-methoxyphenylaminocarbonyl)-6-deoxy-6-azepan-1-yl-α-L-xylo-2-hexulofuranose (Compound No. 194),
2,3-O-Isopropylidene-1-O-decyl-4-O-(4-nitrophenylaminocarbonyl)-6-deoxy-6-azepan-1-yl-α-L-xylo-2-hexulofuranose (Compound No. 195),
2,3-O-Isopropylidene-1-O-heptyl-4-O-(phenylaminocarbonyl)-6-deoxy-6-azepan-1-yl-α-L-xylo-2-hexulofuranose (Compound No. 196),
Hydrochloride salt of 2,3-O-Isopropylidene-1-O-heptyl-4-O-(phenylaminocarbonyl)-6-deoxy-6-azepan-1-yl-α-L-xylo-2-hexulofuranose (Compound No. 197),
2,3-O-Isopropylidene-1-O-heptyl-4-O-(4-chlorophenylaminocarbonyl)-6-deoxy-6-azepan-1-yl-α-L-xylo-2-hexulofuranose (Compound No. 198),
2,3-O-Isopropylidene-1-O-heptyl-4-O-(4-tolylaminocarbonyl)-6-deoxy-6-azepan-1-yl-α-L-xylo-2-hexulofuranose (Compound No. 199),
Hydrochloride salt of 2,3-O-Isopropylidene-1-O-heptyl-4-O-(4-tolylaminocarbonyl)-6-deoxy-6-azepan-1-yl-α-L-xylo-2-hexulofuranose (Compound No. 200),
2,3-O-Isopropylidene-1-O-heptyl-4-O-(4-methoxyphenylaminocarbonyl)-6-deoxy-6-azepan-1-yl-α-L-xylo-2-hexulofuranose (Compound No. 201),
2,3-O-Isopropylidene-1-O-heptyl-4-O-(4-nitrophenylaminocarbonyl)-6-deoxy-6-azepan-1-yl-α-L-xylo-2-hexulofuranose (Compound No. 202),
2,3-O-Isopropylidene-1-O-dodecyl-4-O-(phenylaminocarbonyl)-6-deoxy-6-[2-(pyrrolidin-1-yl)ethyl]amino-α-L-xylo-2-hexulofuranose (Compound No. 203),
2,3-O-Isopropylidene-1-O-dodecyl-4-O-(4-chlorophenylaminocarbonyl)-6-deoxy-6-[2-(pyrrolidin-1-yl)ethyl]amino-α-L-xylo-2-hexulofuranose (Compound No 204),
2,3-O-Isopropylidene-1-O-dodecyl-4-O-(4-tolylaminocarbonyl)-6-deoxy-6-[2-(pyrrolidin-1-yl)ethyl]amino-α-L-xylo-2-hexulofuranose (Compound No. 205),
2,3-O-Isopropylidene-1-O-dodecyl-4-O-(4-methoxyphenylaminocarbonyl)-6-deoxy-6-[2-(pyrrolidin-1-yl)ethyl]amino-α-L-xylo-2-hexulofuranose (Compound No. 206),
2,3-O-Isopropylidene-1-O-dodecyl-4-O-(4-nitrophenylaminocarbonyl)-6-deoxy-6-[2-(pyrrolidin-1-yl)ethyl]amino-α-L-xylo-2-hexulofuranose (Compound No. 207),
2,3-O-Isopropylidene-1-O-decyl-4-O-(phenylaminocarbonyl)-6-deoxy-6-[2-(pyrrolidin-1-yl)ethyl]amino-α-L-xylo-2-hexulofuranose (Compound No. 208),
2,3-O-Isopropylidene-1-O-decyl-4-O-(4-chlorophenylaminocarbonyl)-6-deoxy-6-[2-(pyrrolidin-1-yl)ethyl]amino-α-L-xylo-2-hexulofuranose (Compound No. 209),
2,3-O-Isopropylidene-1-O-decyl-4-O-(4-tolylaminocarbonyl)-6-deoxy-6-[2-(pyrrolidin-1-yl)ethyl]amino-α-L-xylo-2-hexulofuranose (Compound No. 210),
2,3-O-Isopropylidene-1-O-decyl-4-O-(4-methoxyphenylaminocarbonyl)-6-deoxy-6-[2-(pyrrolidin-1-yl)ethyl]amino-α-L-xylo-2-hexulofuranose (Compound No. 211),
2,3-O-Isopropylidene-1-O-decyl-4-O-(4-nitrophenylaminocarbonyl)-6-deoxy-6-[2-(pyrrolidin-1-yl)ethyl]amino-α-L-xylo-2-hexulofuranose (Compound No. 212),
2,3-O-Isopropylidene-1-O-heptyl-4-O-(phenylaminocarbonyl)-6-deoxy-6-[2-(pyrrolidin-1-yl)ethyl]amino-α-L-xylo-2-hexulofuranose (Compound No. 213),
2,3-O-Isopropylidene-1-O-heptyl-4-O-(4-chlorophenylaminocarbonyl)-6-deoxy-6-[2-(pyrrolidin-1-yl)ethyl]amino-α-L-xylo-2-hexulofuranose (Compound No. 214),
2,3-O-Isopropylidene-1-O-heptyl-4-O-(4-tolylaminocarbonyl)-6-deoxy-6-[2-(pyrrolidin-1-yl)ethyl]amino-α-L-xylo-2-hexulofuranose (Compound No. 215),
2,3-O-Isopropylidene-1-O-heptyl-4-O-(4-methoxyphenylaminocarbonyl)-6-deoxy-6-[2-(pyrrolidin-1-yl)ethyl]amino-α-L-xylo-2-hexulofuranose (Compound No. 216),
2,3-O-Isopropylidene-1-O-heptyl-4-O-(4-nitrophenylaminocarbonyl)-6-deoxy-6-[2-(pyrrolidin-1-yl)ethyl]amino-α-L-xylo-2-hexulofuranose (Compound No. 217),
2,3-O-Isopropylidene-1-O-dodecyl-4-O-(phenylaminocarbonyl)-6-deoxy-6-[2-(piperidin-1-yl)ethyl]amino-α-L-xylo-2-hexulofuranose (Compound No. 218),
2,3-O-Isopropylidene-1-O-dodecyl-4-O-(4-chlorophenylaminocarbonyl)-6-deoxy-6-[2-(piperidin-1-yl)ethyl]amino-α-L-xylo-2-hexulofuranose (Compound No. 219),
2,3-O-Isopropylidene-1-O-dodecyl-4-O-(4-tolylaminocarbonyl)-6-deoxy-6-[2-(piperidin-1-yl)ethyl]amino-α-L-xylo-2-hexulofuranose (Compound No. 220),
2,3-O-Isopropylidene-1-O-dodecyl-4-O-(4-methoxyphenylaminocarbonyl)-6-deoxy-6-[2-(piperidin-1-yl)ethyl]amino-α-L-xylo-2-hexulofuranose (Compound No. 221),
2,3-O-Isopropylidene-1-O-dodecyl-4-O-(4-nitrophenylaminocarbonyl)-6-deoxy-6-[2-(piperidin-1-yl)ethyl]amino-α-L-xylo-2-hexulofuranose (Compound No. 222),
2,3-O-Isopropylidene-1-O-decyl-4-O-(phenylaminocarbonyl)-6-deoxy-6-[2-(piperidin-1-yl)ethyl]amino-α-L-xylo-2-hexulofuranose (Compound No. 223),
2,3-O-Isopropylidene-1-O-decyl-4-O-(4-chlorophenylaminocarbonyl)-6-deoxy-6-[2-(piperidin-1-yl)ethyl]amino-α-L-xylo-2-hexulofuranose (Compound No. 224),
2,3-O-Isopropylidene-1-O-decyl-4-O-(4-tolylaminocarbonyl)-6-deoxy-6-[2-(piperidin-1-yl)ethyl]amino-α-L-xylo-2-hexulofuranose (Compound No. 225),
2,3-O-Isopropylidene-1-O-decyl-4-O-(4-methoxyphenylaminocarbonyl)-6-deoxy-6-[2-(piperidin-1-yl)ethyl]amino-α-L-xylo-2-hexulofuranose (Compound No. 226),
2,3-O-Isopropylidene-1-O-decyl-4-O-(4-nitrophenylaminocarbonyl)-6-deoxy-6-[2-(piperidin-1-yl)ethyl]amino-α-L-xylo-2-hexulofuranose (Compound No. 227),
2,3-O-Isopropylidene-1-O-heptyl-4-O-(phenylaminocarbonyl)-6-deoxy-6-[2-(piperidin-1-yl)ethyl]amino-α-L-xylo-2-hexulofuranose (Compound No. 228),
2,3-O-Isopropylidene-1-O-heptyl-4-O-(4-chlorophenylaminocarbonyl)-6-deoxy-6-[2-(piperidin-1-yl)ethyl]amino-α-L-xylo-2-hexulofuranose (Compound No. 229),
2,3-O-Isopropylidene-1-O-heptyl-4-O-(4-tolylaminocarbonyl)-6-deoxy-6-[2-(piperidin-1-yl)ethyl]amino-α-L-xylo-2-hexulofuranose (Compound No. 230),
2,3-O-Isopropylidene-1-O-heptyl-4-O-(4-methoxyphenylaminocarbonyl)-6-deoxy-6-[2-(piperidin-1-yl)ethyl]amino-α-L-xylo-2-hexulofuranose (Compound No. 231),
2,3-O-Isopropylidene-1-O-heptyl-4-O-(4-nitrophenylaminocarbonyl)-6-deoxy-6-[2-(piperidin-1-yl)ethyl]amino-α-L-xylo-2-hexulofuranose (Compound No. 232),
2,3-O-Isopropylidene-1-O-dodecyl-4-O-(phenylaminocarbonyl)-6-deoxy-6-[2-(morphilin-4-yl)ethyl]amino-α-L-xylo-2-hexulofuranose (Compound No. 233),
2,3-O-Isopropylidene-1-O-dodecyl-4-O-(4-chlorophenylaminocarbonyl)-6-deoxy-6-(2-ethylmorphilin-4-yl)-α-L-xylo-2-hexulofuranose (Compound No. 234),
2,3-O-Isopropylidene-1-O-dodecyl-4-O-(4-tolylaminocarbonyl)-6-deoxy-6-[2-(morphilin-4-yl)ethyl]amino-α-L-xylo-2-hexulofuranose (Compound No. 235),
2,3-O-Isopropylidene-1-O-dodecyl-4-O-(4-methoxyphenylaminocarbonyl)-6-deoxy-6-[2-(morphilin-4-yl)ethyl]amino-α-L-xylo-2-hexulofuranose (Compound No. 236),
2,3-O-Isopropylidene-1-O-dodecyl-4-O-(4-nitrophenylaminocarbonyl)-6-deoxy-6-[2-(morphilin-4-yl)ethyl]amino-α-L-xylo-2-hexulofuranose (Compound No. 237),
2,3-O-Isopropylidene-1-O-decyl-4-O-(phenylaminocarbonyl)-6-deoxy-6-[2-(morphilin-4-yl)ethyl]amino-α-L-xylo-2-hexulofuranose (Compound No. 238),
2,3-O-Isopropylidene-1-O-decyl-4-O-(4-chlorophenylaminocarbonyl)-6-deoxy-6-[2-(morphilin-4-yl)ethyl]amino-α-L-xylo-2-hexulofuranose (Compound No. 239),
2,3-O-Isopropylidene-1-O-decyl-4-O-(4-tolylaminocarbonyl)-6-deoxy-6-[2-(morphilin-4-yl)ethyl]amino-α-L-xylo-2-hexulofuranose (Compound No. 240),
Hydrochloride salt of 2,3-O-Isopropylidene-1-O-decyl-4-O-(4-tolylaminocarbonyl)-6-deoxy-6-[2-(morphilin-4-yl)ethyl]amino-α-L-xylo-2-hexulofuranose (Compound No. 241),
2,3-O-Isopropylidene-1-O-decyl-4-O-(4-methoxyphenylaminocarbonyl)-6-deoxy-6-[2-(morphilin-4-yl)ethyl]amino-α-L-xylo-2-hexulofuranose (Compound No. 242),
2,3-O-Isopropylidene-1-O-dodecyl-4-O-(4-nitrophenylaminocarbonyl)-6-deoxy-6-[2-(morphilin-4-yl)ethyl]amino-α-L-xylo-2-hexulofuranose (Compound No. 243),
2,3-O-Isopropylidene-1-O-heptyl-4-O-(phenylaminocarbonyl)-6-deoxy-6-[2-(morphilin-4-yl)ethyl]amino-α-L-xylo-2-hexulofuranose (Compound No. 244),
2,3-O-Isopropylidene-1-O-heptyl-4-O-(4-chlorophenylaminocarbonyl)-6-deoxy-6-[2-(morphilin-4-yl)ethyl]amino-α-L-xylo-2-hexulofuranose (Compound No. 245),
2,3-O-Isopropylidene-1-O-heptyl-4-O-(4-tolylaminocarbonyl)-6-deoxy-6-[2-(morphilin-4-yl)ethyl]amino-α-L-xylo-2-hexulofuranose (Compound No. 246),
2,3-O-Isopropylidene-1-O-heptyl-4-O-(4-methoxyphenylaminocarbonyl)-6-deoxy-6-[2-(morphilin-4-yl)ethyl]amino-α-L-xylo-2-hexulofuranose (Compound No. 247),
2,3-O-Isopropylidene-1-O-heptyl-4-O-(4-nitrophenylaminocarbonyl)-6-deoxy-6-[2-(morphilin-4-yl)ethyl]amino-α-L-xylo-2-hexulofuranose (Compound No. 248),
2,3,O-Isopropylidene-1-O-dodecyl-4-O-(methylaminocarbonyl)-6-deoxy-6-azepan-1-yl-α-L-xylo-2-hexulofuranose (Compound No. 249),
2,3,O-Isopropylidene-1-O-dodecyl-4-O-(methylaminocarbonyl)-6-deoxy-6-morpholin-4-yl-α-L-xylo-2-hexulofuranose (Compound No. 250),
2,3,O-Isopropylidene-1-O-dodecyl-4-O-(methylaminocarbonyl)-6-deoxy-6-piperidin-1-yl-α-L-xylo-2-hexulofuranose (Compound No. 251),
2,3,O-Isopropylidene-1-O-dodecyl-4-O-(methylaminocarbonyl)-6-deoxy-6-[2-(pyrrolidin-1-yl)ethyl]amino-α-L-xylo-2-hexulofuranose (Compound No. 252),
2,3,O-Isopropylidene-1-O-dodecyl-4-O-(methylaminocarbonyl)-6-deoxy-6-[2-(morphilin-4-yl)ethyl]amino-α-L-xylo-2-hexulofuranose (Compound No. 253),
2,3,O-Isopropylidene-1-O-decyl-4-O-(methylaminocarbonyl)-6-deoxy-6-morpholin-4-yl-α-L-xylo-2-hexulofuranose (Compound No. 254),
2,3,O-Isopropylidene-1-O-decyl-4-O-(methylaminocarbonyl)-6-deoxy-6-azepan-1-yl-α-L-xylo-2-hexulofuranose (Compound No. 255),
2,3,O-Isopropylidene-1-O-decyl-4-O-(methylaminocarbonyl)-6-deoxy-6-piperidin-1-yl-α-L-xylo-2-hexulofuranose (Compound No. 256),
2,3,O-Isopropylidene-1-O-decyl-4-O-(methylaminocarbonyl)-6-deoxy-6-[2-(pyrrolidin-1-yl)ethyl]amino-α-L-xylo-2-hexulofuranose (Compound No. 257),
2,3,O-Isopropylidene-1-O-decyl-4-O-(methylaminocarbonyl)-6-deoxy-6-[2-(morphilin-4-yl)ethyl]amino-α-L-xylo-2-hexulofuranose (Compound No. 258),
2,3,O-Isopropylidene-1-O-heptyl-4-O-(methylaminocarbonyl)-6-deoxy-6-pyrrolidin-1-yl-α-L-xylo-2-hexulofuranose (Compound No. 259),
2,3,O-Isopropylidene-1-O-heptyl-4-O-(methylaminocarbonyl)-6-deoxy-6-morpholin-4-yl-α-L-xylo-2-hexulofuranose (Compound No. 260),
2,3,O-Isopropylidene-1-O-heptyl-4-O-(methylaminocarbonyl)-6-deoxy-6-azepan-1-yl-α-L-xylo-2-hexulofuranose (Compound No. 261),
2,3,O-Isopropylidene-1-O-heptyl-4-O— (methylaminocarbonyl)-6-deoxy-6-piperidin-1-yl-α-L-xylo-2-hexulofuranose (Compound No. 262),
2,3,O-Isopropylidene-1-O-heptyl-4-O-(methylaminocarbonyl)-6-deoxy-6-[2-(pyrrolidin-1-yl)ethyl]amino-α-L-xylo-2-hexulofuranose (Compound No. 263), and
2,3,O-Isopropylidene-1-O-heptyl-4-O-(methylaminocarbonyl)-6-deoxy-6-[2-(morphilin-4-yl)ethyl]amino-α-L-xylo-2-hexulofuranose (Compound No. 264).

### Pharmacological Activity

The compounds of Formulae I, II, III and IV showed inhibitory activity on A23187-induced LTB4 release from human neutrophils. Standard assays were performed on particular illustrative compounds of Formulae I, II, III and Formula IV, and are mentioned below.

### A23187 Induced LTB4 Release from Human Neutrophils

Neutrophils were isolated from freshly drawn human blood after dextran sedimentation and ficoll separation (Eur J. Biochem., 169, 175, 1987). 180 μl of the neutrophil suspension (0.2×10⁶ cells/ml) were taken, and added 19 μl of Hank's Buffer salt solution along with 1 μl of the test drug (200 times concentrated) in a 24-well plate and incubated at 37° C. for 1 hr. 3 min before the end of test compound incubation, 0.25 mM Ca⁺⁺/Mg⁺⁺ was added. Then, 0.3 μg/ml A23187 was added and incubated for 10 min further. The reaction was stopped by adding 80 μl of cold methanol and spun at 3500 rpm for 10 min. to remove cell debris. The samples were analysed for LTB₄ assay (J. Pharmacol. Exp. Ther. 297:267, 2001) using LTB₄ ELISA kits (Assay Design Inc., USA). The amount of LTB₄ was quantified and percent inhibition in LTB₄ release was calculated with respect to negative and positive controls to compute IC₅₀ values.

Compound Nos. 1, 2, 6, 10, 14, 33, 36, 79, 80, 83-85, 87, 92, 99, 101, 102, 105, 110, 115, 120, 168, 177, 193, 194, 197, 200, 210, and 241 were tested, giving IC₅₀ val—of between about 2.5 μm and abμm out 30 μm, or between about 2.5 μm and about 10 μm, or between about 2.5 μm and about 5 μm.

We claim:

1. A method of inhibiting the 5-lipoxygenase enzyme or related processes in a mammal comprising administering to the animal a therapeutically effective amount of a compound having the structure of Formula I, 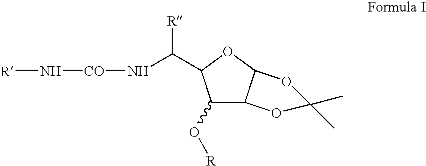
its pharmaceutically acceptable salts, esters, enantiomers, diastereomers, N-oxides, or amides, wherein
R is C₁ to C₁₅ alkyl, alkene, alkyne (straight chain or branched), aryl, substituted aryl or alkyl aryl;
R′ is SO₂C₆H₅, SO₂C₆H₄CH₃-p, or SO₂C₆H₄—Cl-p, phenyl or substituted phenyl, represented as C₆H₄—R′″-p, wherein R′″ is Cl, NO₂, OCH₃, CH₃, CH₂COOH, CH₂COOCH₃, CH₂COLDVP, CH₂CODVP, or CH₂COVP, wherein LDVP DVP and VP represent tetra peptide (Leucyl-aspartyl-alyl-prolyl), tripeptide (aspartyl-valyl-prolyl) and dipeptide (valyl-prolyl) respectively; and
R″ is H or CH₃; wherein [symbol]
represents idofuranose, talofuranose, xylofuranose or ribofuranose configurations.

2. A method of inhibiting the 5-lipoxygenase enzyme or related processes in a mammal comprising administering to the said animal a therapeutically effective amount of a compound having the structure of Formula II, 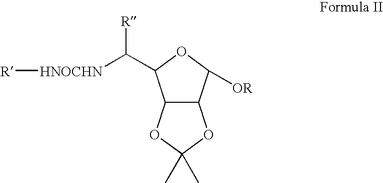
its pharmaceutically acceptable salts, esters, enantiomers, diastereomers, N-oxides, or amides, wherein;
R is C₁ to C₁₅ alkyl, alkene, alkyne (straight chain or branched), aryl, substituted aryl or alkyl aryl;
R′ is SO₂C₆H₅, SO₂C₆H₅CH₃-p, SO₂C₆H₄Cl-p, phenyl or substituted phenyl, represented as C₆H₄—R′″-p, wherein R′″ is Cl, NO₂, OCH₃, CH₃, CH₂COOH, CH₂COOCH₃, CH₂COLDVP, CH₂CODVP, or CH₂COVP, wherein LDVP, DVP and VP represent tetra peptide (Leucyl-aspartyl-valyl-prolyl), tripeptide (aspartyl-valyl-prolyl) and dipeptide (valyl-prolyl), respectively; and
R″ is H or CH₃.

3. A method of inhibiting 5-lipoxygenase enzyme or related processes in a mammal comprising administering to the animal a therapeutically effective amount of a compound having the structure of Formula III, 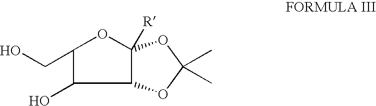
its pharmaceutically acceptable salts, esters, enantiomers, diastereomers, N-oxides, or amides, wherein
R′ is CO-LDVP, CO-DVP, CO—VP or CH₂NHCONHR″ wherein LDVP, DVP and VP represent tetra peptide (Leucyl-aspartyl-valyl-prolyl), tripeptide (aspartyl-valyl-prolyl) and dipeptide (valyl-prolyl), respectively; wherein R″ is C₆H₄R′″-p (R′″ is Cl, NO₂, OCH₃, CH₃, CH₂COOH, CH₂COLDVP, CH₂CODVP or CH₂COVP wherein LDVP, DVP and VP are the same as defined earlier).

4. A method of inhibiting 5-lipoxygenase enzyme or related processes in a mammal comprising administering to the animal a therapeutically effective amount of a compound having the structure of Formula IV, 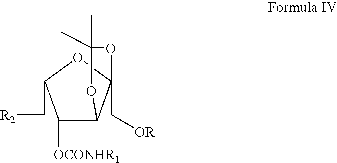
its pharmaceutically acceptable salts, esters, enantiomers, diastereomers, N-oxides, or amides, wherein
R is C₁ to C₁₅ alkyl, alkene, alkyne (straight chain or branched), aryl, substituted aryl or alkyl aryl;
R₁ is methoyl phenyl, o-m- or p-chlorophenyl, tolyl, methoxyphenyl or nitrophenyl; and
R₂ is H, pyrrolidinyl, piperidinyl, morphilinyl or hexamethyleneimino or a radical of the Formula—NHR₃ wherein R₃ is C₁ to C₁₅ alkyl, alkene or alkyne (straight chain or branched) or a radical of the Formula 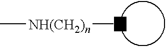
in which n is a whole number in the range of 2-5 and 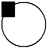
is a five, six- or seven-membered heterocyclic ring containing one or more hetero atoms.

5. A method of inhibiting 5-lipoxygenase enzyme and related processes in a mammal comprising administering to the animal a therapeutically effective amount of a compound selected from the group consisting of:
1,2-O-isopropylidene-3-O-dodecyl-5,6-dideoxy-5-N-{[4-(2-hydroxy-2-oxoethyl)phenyl]aminocarbonyl amino}-β-L-idofuranose,
1,2-O-isopropylidene-3-O-decyl-5,6-dideoxy-5-N-{[4-(2-hydroxy-2-oxoethyl)phenyl]aminocarbonylamino}-β-L-idofuranose,
1,2-O-isopropylidene-3-O-heptyl-5,6-dideoxy-5-N-{[4-(2-hydroxy-2-oxoethyl)phenyl]aminocarbonylamino}-β-L-idofuranose,
1,2-O-isopropylidene-3-O-butyl-5,6-dideoxy-5-N-{[4-(2-hydroxy-2-oxoethyl)phenyl]aminocarbonylamino}-β-L-idofuranose,
1,2-O-isopropylidene-3-O-dodecyl-5,6-dideoxy-5-N-{[4-chlorophenyl]aminocarbonylamino}-β-L-idofuranose,
1,2-O-isopropylidene-3-O-decyl-5,6-dideoxy-5-N-{[4-chlorophenyl]aminocarbonylamino}-β-L-idofuranose,
1,2-O-isopropylidene-3-O-heptyl-5,6-dideoxy-5-N-{[4-chlorophenyl]aminocarbonylamino}-β-L-idofuranose, 1,2-O-isopropylidene-3-O-butyl-5,6-dideoxy-5-N-{[4-chlorophenyl]aminocarbonylamino}-β-L-idofuranose,
1,2-O-isopropylidene-3-O-dodecyl-5,6-dideoxy-5-N-{[4-methoxyphenyl]amino carbonylamino}-β-L-idofuranose,
1,2-O-isopropylidene-3-O-decyl-5,6-dideoxy-5-N-{[4-methoxyphenyl]amino carbonylamino}-β-L-idofuranose,
1,2-O-isopropylidene-3-O-heptyl-5,6-dideoxy-5-N-{[4-methoxyphenyl]amino carbonylamino}-β-L-idofuranose,
1,2-O-isopropylidene-3-O-butyl-5,6-dideoxy-5-N-{[4-methoxyphenyl]aminocarbonylamino}-β-L-idofuranose,
1,2-O-isopropylidene-3-O-dodecyl-5,6-dideoxy-5-N-{[4-tolyl]aminocarbonylamino}-β-L-idofuranose,
1,2-O-isopropylidene-3-O-decyl-5,6-dideoxy-5-N-{[4-tolyl]aminocarbonylamino}-β-L-idofuranose,
1,2-O-isopropylidene-3-O-heptyl-5,6-dideoxy-5-N-{[4-tolyl]aminocarbonylamino}-β-L-idofuranose,
1,2-O-isopropylidene-3-O-butyl-5,6-dideoxy-5-N-{[4-tolyl]aminocarbonylamino}-β-L-idofuranose,
1,2-O-isopropylidene-3-O-dodecyl-5,6-dideoxy-5-N-{[4-(2-hydroxy-2-oxoethyl)phenyl]amino carbonyl amino}-β-L-talofuranose,
1,2-O-isopropylidene-3-O-decyl-5,6-dideoxy-5-N-{[4-(2-hydroxy-2-oxoethyl)phenyl]aminocarbonylamino}-β-L-talofuranose,
1,2-O-isopropylidene-3-O-heptyl-5,6-dideoxy-5-N-{[4-(2-hydroxy-2-oxoethyl)phenyl]aminocarbonylamino}-β-L-talofuranose,
1,2-O-isopropylidene-3-O-butyl-5,6-dideoxy-5-N-{[4-(2-hydroxy-2-oxoethyl)phenyl]aminocarbonyl amino}-β-L-talofuranose,
1,2-O-isopropylidene-3-O-dodecyl-5,6-dideoxy-5-N-{[4-chlorophenyl]amino carbonylamino}-β-L-talofuranose,
1,2-O-isopropylidene-3-O-decyl-5,6-dideoxy-5-N-{[4-chlorophenyl]aminocarbonylamino}α,D-5-epiallofuranose,
1,2-O-isopropylidene-3-O-heptyl-5,6-dideoxy-5-N-{[4-chlorophenyl]aminocarbonylamino}-β-L-talofuranose,
1,2-O-isopropylidene-3-O-butyl-5,6-dideoxy-5-N-{[4-chlorophenyl]aminocarbonylamino}-β-L-talofuranose,
1,2-O-isopropylidene-3-O-dodecyl-5,6-dideoxy-5-N-{[4-methoxyphenyl]amino carbonylamino}-β-L-talofuranose,
1,2-O-isopropylidene-3-O-decyl-5,6-dideoxy-5-N-{[4-methoxyphenyl]aminocarbonylamino}-β-L-talofuranose,
1,2-O-isopropylidene-3-O-heptyl-5,6-dideoxy-5-N-{[4-methoxyphenyl]aminocarbonylamino}-β-L-talofuranose,
1,2-O-isopropylidene-3-O-butyl-5,6-dideoxy-5-N-{[4-methoxyphenyl]amino carbonylamino}-β-L-talofuranose,
1,2-O-isopropylidene-3-O-dodecyl-5,6-dideoxy-5-N-{[4-tolyl]aminocarbonylamino}-β-L-talofuranose,
1,2-O-isopropylidene-3-O-decyl-5,6-dideoxy-5-N-{[4-tolyl]aminocarbonylamino}-β-L-talofuranose,
1,2-O-isopropylidene-3-O-heptyl-5,6-dideoxy-5-N-{[4-tolyl]aminocarbonylamino}-β-L-talofuranose,
1,2-O-isopropylidene-3-O-butyl-5,6-dideoxy-5-N-{[4-tolyl]aminocarbonyl amino}-β-L-talofuranose,
1,2-O-isopropylidene-3-O-dodecyl-5-deoxy-5-N-{[4-(2-hydroxy-2-oxoethyl)phenyl]aminocarbonylamino}-α,D-xylofuranose,
1,2-O-isopropylidene-3-O-decyl-5-deoxy-5-N-{[4-(2-hydroxy-2-oxoethyl)phenyl]aminocarbonylamino}-α,D-xylofuranose,
1,2-O-isopropylidene-3-O-hexyl-5-deoxy-5-N-{[4-(2-hydroxy-2-oxoethyl)phenyl]aminocarbonylamino}-α,D-xylofuranose,
1,2-O-isopropylidene-3-O-butyl-5-deoxy-5-N-{[4-(2-hydroxy-2-oxoethyl)phenyl]aminocarbonylamino}-α,D-xylofuranose,
1,2-O-isopropylidene-3-O-methyl-5-deoxy-5-N-{[4-(2-hydroxy-2-oxoethyl)phenyl]aminocarbonylamino}-α,D-xylofuranose,
1,2-O-isopropylidene-3-O-dodecyl-5-deoxy-5-N-{[4-chlorophenyl]amino carbonylamino}1α,D-xylofuranose,
1,2-O-isopropylidene-3-O-decyl-5-deoxy-5-N-{[4-chlorophenyl]amino carbonylamino}1α,D-xylofuranose,
1,2-O-isopropylidene-3-O-hexyl-5-deoxy-5-N-{[4-chlorophenyl]amino carbonylamino}1α,D-xylofuranose,
1,2-O-isopropylidene-3-O-butyl-5-deoxy-5-N-{[4-chlorophenyl]amino carbonylamino}1α,D-xylofuranose,
1,2-O-isopropylidene-3-O-methyl-5-deoxy-5-N-{[4-chlorophenyl]amino carbonylamino}1α,D-xylofuranose,
1,2-O-isopropylidene-3-O-dodecyl-5-deoxy-5-N-{[4-methoxyphenyl]amino carbonylamino}1α,D-xylofuranose,
1,2-O-isopropylidene-3-O-decyl-5-deoxy-5-N-{[4-methoxyphenyl]aminocarbonylamino}1α,D-xylofuranose,
1,2-O-isopropylidene-3-O-hexyl-5-deoxy-5-N-{[4-methoxyphenyl]aminocarbonylamino}1α,D-xylofuranose,
1,2-O-isopropylidene-3-O-butyl-5-deoxy-5-N-{[4-methoxyphenyl]aminocarbonylamino}1α,D-xylofuranose,
1,2-O-isopropylidene-3-O-methyl-5-deoxy-5-N-{[4-methoxyphenyl]aminocarbonylamino}1α,D-xylofuranose,
1,2-O-isopropylidene-3-O-dodecyl-5-deoxy-5-N-{[4-tolyl]aminocarbonylamino}α,D-xylofuranose,
1,2-O-isopropylidene-3-O-decyl-5-deoxy-5-N-{[4-tolyl]aminocarbonylamino}α,D-xylofuranose,
1,2-O-isopropylidene-3-O-hexyl-5-deoxy-5-N-{[4-tolyl]aminocarbonylamino}α,D-xylofuranose,
1,2-O-isopropylidene-3-O-butyl-5-deoxy-5-N-{[4-tolyl]aminocarbonylamino}α,D-xylofuranose,
1,2-O-isopropylidene-3-O-methyl-5-deoxy-5-N-{[4-tolyl]aminocarbonylamino}α,D-xylofuranose,
1,2-O-isopropylidene-3-O-dodecyl-5-deoxy-5-N-{[aminocarbonylamino]phenyl acetyl-L-Leucyl-α,L-Aspartyl-L-Valyl-L-Proline}-α,D-xylofuranose,
1,2-O-isopropylidene-3-O-dodecyl-5-deoxy-5-N-{[aminocarbonylamino]phenylacetyl-α,L-Aspartyl-L-Valyl-L-Proline}-α,D-xylofuranose,
1,2-O-isopropylidene-3-O-dodecyl-5-deoxy-5-N-{[aminocarbonylamino]phenylacetyl-alyl-L-Proline}-α,D-xylofuranose,
1,2-O-isopropylidene-3-O-dodecyl-5-deoxy-5-N-{[4-(2-hydroxy-2-oxoethyl)phenyl]aminocarbonylamino}-α,D-ribofuranose,
1,2-O-isopropylidene-3-O-decyl-5-deoxy-5-N-{[4-(2-hydroxy-2-oxoethyl)phenyl]aminocarbonylamino}-α,D-ribofuranose,
1,2-O-isopropylidene-3-O-heptyl-5-deoxy-5-N-{[4-(2-hydroxy-2-oxoethyl)phenyl]aminocarbonylamino}-α,D-ribofuranose,
1,2-O-isopropylidene-3-O-butyl-5-deoxy-5-N-{[4-(2-hydroxy-2-oxoethyl)phenyl]aminocarbonylamino}-α,D-ribofuranose,
1,2-O-isopropylidene-3-O-methyl-5-deoxy-5-N-{[4-(2-hydroxy-2-oxoethyl)phenyl]aminocarbonylamino}-α,D-ribofuranose,
1,2-O-isopropylidene-3-O-dodecyl-5-deoxy-5-N-{[4-chlorophenyl]amino carbonylamino}α,D-ribofuranose,
1,2-O-isopropylidene-3-O-decyl-5-deoxy-5-N-{[4-chlorophenyl]aminocarbonylamino}1α,D-ribofuranose,
1,2-O-isopropylidene-3-O-heptyl-5-deoxy-5-N-{[4-chlorophenyl]aminocarbonylamino}1α,D-ribofuranose,
1,2-O-isopropylidene-3-O-butyl-5-deoxy-5-N-{[4-chlorophenyl]aminocarbonylamino}1α,D-ribofuranose,
1,2-O-isopropylidene-3-O-methyl-5-deoxy-5-N-{[4-chlorophenyl]aminocarbonylamino}1α,D-ribofuranose,
1,2-O-isopropylidene-3-O-dodecyl-5-deoxy-5-N-{[4-methoxyphenyl]amino carbonylamino}1α,D-ribofuranose,
1,2-O-isopropylidene-3-O-decyl-5-deoxy-5-N-{[4-methoxyphenyl]aminocarbonylamino}1α,D-ribofuranose,
1,2-O-isopropylidene-3-O-heptyl-5-deoxy-5-N-{[4-methoxyphenyl]amino carbonylamino}1α,D-ribofuranose,
1,2-O-isopropylidene-3-O-butyl-5-deoxy-5-N-{[4-methoxyphenyl]aminocarbonylamino}α,D-ribofuranose,
1,2-O-isopropylidene-3-O-methyl-5-deoxy-5-N-{[4-methoxyphenyl]amino carbonylamino}α,D-ribofuranose,
1,2-O-isopropylidene-3-O-dodecyl-5-deoxy-5-N-{[4-tolyl]aminocarbonylamino}α,D-ribofuranose,
1,2-O-isopropylidene-3-O-decyl-5-deoxy-5-N-{[4-tolyl]aminocarbonylamino}α, D-ribofuranose,
1,2-O-isopropylidene-3-O-heptyl-5-deoxy-5-N-{[4-tolyl]aminocarbonylamino}α,D-ribofuranose,
1,2-O-isopropylidene-3-O-butyl-5-deoxy-5-N-{[4-tolyl]aminocarbonylamino}α, D-ribofuranose,
1,2-O-isopropylidene-3-O-methyl-5-deoxy-5-N-{[4-olyl]aminocarbonylamino}α, D-ribofuranose,
1,2-O-isopropylidene-3-O-dodecyl-5-deoxy-5-N-{[aminocarbonylaminophenyl]acetyl-L-Leucyl-α,L-Aspartyl-L-Valyl-L-Proline}-α,D-ribofuranose,
1,2-O-isopropylidene-3-O-dodecyl-5-deoxy-5-N-{[aminocarbonylaminophenyl]acetyl-α,L-Aspartyl-L-Valyl-L-Proline}-α,D-ribofuranose,
1,2-O-isopropylidene-3-O-dodecyl-5-deoxy-5-N-[aminocarbonylamino phenyl]acetyl-L-Valyl-L-Proline}-α,D-ribofuranose.

6. A method of inhibiting 5-lipoxygenase enzyme or related processes in a mammal comprising administering to the said animal a therapeutically effective amount of a compound selected from the group consisting of:
2,3-O-isopropylidene-1-O-butyl-5,6-dideoxy-5-N-{[4-(2-hydroxy-2-oxoethyl)phenyl]aminocarbonyl amino}-β,L-gulofuranoside,
2,3-O-isopropylidene-1-O-hexyl-5,6-dideoxy-5-N-{[4-(2-hydroxy-2-oxoethyl)phenyl]aminocarbonyl amino}-β,L-gulofuranoside,
2,3-O-isopropylidene-1-O-heptyl-5,6-dideoxy-5-N-{[4-(2-hydroxy-2-oxoethyl)phenyl]aminocarbonyl amino}-β,L-gulofuranoside,
2,3-O-isopropylidene-1-O-decyl-5,6-dideoxy-5-N-{[4-(2-hydroxy-2-oxoethyl)phenyl]aminocarbonyl amino}-β,L-gulofuranoside,
2,3-O-isopropylidene-1-O-dodecyl-5,6-dideoxy-5-N-{[4-(2-hydroxy-2-oxoethyl)phenyl]aminocarbonylamino}-β,L-gulofuranoside,
Ditris salt of 2,3-O-isopropylidene-1-O-dodecyl-5,6-dideoxy-5-N-{[4-(2-hydroxy-2-oxoethyl)phenyl]aminocarbonylamino}β,L-gulofuranoside,
Sodium salt of 2,3-O-isopropylidene-1-O-dodecyl-5,6-dideoxy-5-N-{[4-(2-hydroxy-2-oxoethyl)phenyl]aminocarbonylamino}β,L-gulofuranoside,
2,3-O-isopropylidene-1-O-butyl-5,6-dideoxy-5-N-{[4-chlorophenyl]aminocarbonylamino}-β,L-gulofuranoside,
2,3-O-isopropylidene-1-O-hexyl-5,6-dideoxy-5-N-{[4-chlorophenyl]amino carbonylamino}-β,L-gulofuranoside,
2,3-O-isopropylidene-1-O-heptyl-5,6-dideoxy-5-N-{[4-chlorophenyl]amino carbonylamino}-β,L-gulofuranoside,
2,3-O-isopropylidene-1-O-decyl-5,6-dideoxy-5-N-{[4-chlorophenyl]amino carbonylamino}-β,L-gulofuranoside,
2,3-O-isopropylidene-1-O-dodecyl-5,6-dideoxy-5-N-{[4-chlorophenyl]amino carbonylamino}-β,L-gulofuranoside,
2,3-O-isopropylidene-1-O-butyl-5,6-dideoxy-5-N-{[4-methoxyphenyl]amino carbonylamino}-β,L-gulofuranoside,
2,3-O-isopropylidene-1-O-hexyl-5,6-dideoxy-5-N-{[4-methoxyphenyl]amino carbonylamino}-β,L-gulofuranoside,
2,3-O-isopropylidene-1-O-heptyl-5,6-dideoxy-5-N-{[4-methoxyphenyl]amino carbonylamino}-β,L-gulofuranoside,
2,3-O-isopropylidene-1-O-decyl-5,6-dideoxy-5-N-{[4-methoxyphenyl]amino carbonylamino}-β,L-gulofuranoside,
2,3-O-isopropylidene-1-O-dodecyl-5,6-dideoxy-5-N-{[4-methoxyphenyl]amino carbonylamino}-β,L-gulofuranoside,
2,3-O-isopropylidene-1-O-butyl-5,6-dideoxy-5-N-{[4-tolyl]amino carbonylamino}-β,L-gulofuranoside,
2,3-O-isopropylidene-1-O-hexyl-5,6-dideoxy-5-N-{[4-tolyl]aminocarbonylamino}-β,L-gulofuranoside,
2,3-O-isopropylidene-1-O-heptyl-5,6-dideoxy-5-N-{[4-tolyl]aminocarbonylamino}-β,L-gulofuranoside,
2,3-O-isopropylidene-1-O-decyl-5,6-dideoxy-5-N-{[4-tolyl]aminocarbonylamino}-β,L-gulofuranoside,
2,3-O-isopropylidene-1-O-dodecyl-5,6-dideoxy-5-N-{[4-tolyl]aminocarbonylamino}-β,L-gulofuranoside,
2,3-O-isopropylidene-1-O-methyl-5-deoxy-5-N-{[4-(2-hydroxy-2-oxoethyl)phenyl]aminocarbonylamino}-α,D-lyxofuranoside,
2,3-O-isopropylidene-1-O-butyl-5-deoxy-5-N-{[4-(2-hydroxy-2-oxoethyl)phenyl]aminocarbonylamino}-α,D-lyxofuranoside,
2,3-O-isopropylidene-1-O-heptyl-5-deoxy-5-N-{[4-(2-hydroxy-2-oxoethyl)phenyl]aminocarbonylamino}-α,D-lyxofuranoside,
2,3-O-isopropylidene-1-O-decyl-5-deoxy-5-N-{[4-(2-hydroxy-2-oxoethyl)phenyl]aminocarbonylamino}-α,D-lyxofuranose,
2,3-O-isopropylidene-1-O-dodecyl-5-deoxy-5-N-{[4-(2-hydroxy-2-oxoethyl)phenyl]aminocarbonyl amino}-α,D-lyxofuranoside,
2,3-O-isopropylidene-1-O-methyl-5-deoxy-5-N-{[4-chlorophenyl]aminocarbonylamino}1α,D-lyxofuranoside,
2,3-O-isopropylidene-1-O-butyl-5-deoxy-5-N-{[4-chlorophenyl]aminocarbonylamino}α,D-lyxofuranoside,
2,3-O-isopropylidene-1-O-heptyl-5-deoxy-5-N-{[4-chlorophenyl]aminocarbonylamino}1β,D-lyxofuranoside,
2,3-O-isopropylidene-1-O-decyl-5-deoxy-5-N-{[4-chlorophenyl]aminocarbonylamino}1β,D-lyxofuranoside,
2,3-O-isopropylidene-1-O-dodecyl-5-deoxy-5-N-{[4-chlorophenyl]amino carbonylamino}1β,D-lyxofuranoside,
2,3-O-isopropylidene-1-O-methyl-5-deoxy-5-N-{[4-methoxyphenyl]amino carbonylamino}1β,D-lyxofuranoside,
2,3-O-isopropylidene-1-O-butyl-5-deoxy-5-N-{[4-methoxyphenyl]aminocarbonylamino}1β,D-lyxofuranoside,
2,3-O-isopropylidene-1-O-heptyl-5-deoxy-5-N-{[4-methoxyphenyl]amino carbonylamino}1α,D-lyxofuranoside,
2,3-O-isopropylidene-1-O-decyl-5-deoxy-5-N-{[4-methoxyphenyl]aminocarbonylamino}α,D-lyxofuranoside,
2,3-O-isopropylidene-1-O-dodecyl-5-deoxy-5-N-{[4-methoxyphenyl]amino carbonylamino}αD-lyxofuranoside,
2,3-O-isopropylidene-1-O-methyl-5-deoxy-5-N-{[4-tolyl]amino carbonylamino}α,D-lyxofuranoside,
2,3-O-isopropylidene-1-O-butyl-5-deoxy-5-N-{[4-tolyl]aminocarbonylamino}α, D-lyxofuranoside,
2,3-O-isopropylidene-1-O-heptyl-5-deoxy-5-N-{[4-tolyl]aminocarbonylamino}α, D-lyxofuranoside,
2,3-O-isopropylidene-1-O-decyl-5-deoxy-5-N-{[4-tolyl]aminocarbonylamino}α, D-lyxofuranoside,
2,3-O-isopropylidene-1-O-dodecyl-5-deoxy-5-N-{[4-tolyl]amino carbonylamino}α,D-lyxofuranoside,
2,3-O-isopropylidene-1-O-dodecyl-5-deoxy-5-N-{[aminocarbonylamino]phenylacetyl-L-Leucyl-α,L-Aspartyl-L-Valyl-L-Proline}-α,D-lyxofuranoside,
2,3-O-isopropylidene-1-O-dodecyl-5-deoxy-5-N-{[aminocarbonylamino]phenyl acetyl-α,L-Aspartyl-L-Valyl-L-Proline}-α,D-lyxofuranoside,
2,3-O-isopropylidene-1-O-dodecyl-5-deoxy-5-N-{[aminocarbonylamino]phenyl acetyl-L-Valyl-L-Proline}-α,D-lyxofuranoside.

7. A method of inhibiting 5-lipoxygenase enzyme or related processes in a mammal comprising administering to the animal a therapeutically effective amount of a compound selected from the group consisting of:
2,3;4,6-Di-O-isopropylidene-1-carbonyl-L-Leucyl-α,L-Aspartyl-L-Valyl-L-Proline-α,L-xylo-2-hexulofuranosonic acid,
2,3;4,6-Di-O-isopropylidene-1-carbonyl-α,L-Aspartyl-L-Valyl-L-Proline-α,L-xylo-2-hexulofuranosonic acid,
2,3-O-isopropylidene-1-carbonyl-L-Leucyl-α,L-Aspartyl-L-Valyl-L-Proline-α,L-xylo-2-hexulofuranosonic acid,
2,3-O-isopropylidene-1-carbonyl-α,L-Aspartyl-L-Valyl-L-Proline-α,L-xylo-2-hexulofuranosonic acid,
2,3-O-isopropylidene-1-carbonyl-L-Valyl-L-Proline-α,L-xylo-2-hexulofuranosonic acid,
2,3;4,6-Di-O-isopropylidene-1-deoxy-1-N-{[4-chlorophenyl]aminocarbonylamino}-α,L-xylo-2-hexulofuranose,
2,3;4,6-Di-O-isopropylidene-1-deoxy-1-N-{[4-methoxyphenyl]aminocarbonylamino}-α,L-xylo-2-hexulofuranose,
2,3;4,6-Di-O-isopropylidene-1-deoxy-1-N-{[4-tolyl]aminocarbonylamino}-α,L-xylo-2-hexulofuranose,
2,3-O-isopropylidene-1-deoxy-1-N-{[4-(2-hydroxy-2oxoethyl)phenyl]aminocarbonylamino}-α,L-xylo-2-hexulofuranose,
2,3-O-isopropylidene-1-deoxy-1-N-{[4-chlorophenyl]aminocarbonylamino}-α,L-xylo-2-hexulofuranose,
2,3-O-isopropylidene-1-deoxy-1-N-{[4-methoxyphenyl]aminocarbonylamino}-α,L-xylo-2-hexulofuranose,
2,3-O-isopropylidene-1-deoxy-1-N-{[4-tolyl]aminocarbonylamino}-α,L-xylo-2-hexulofuranose,
2,3-O-isopropylidene-1 deoxy-1-N-{[aminocarbonylaminophenylacetyl-L-Leucyl-α,L-Aspartyl-L-Valyl-L-Proline)}-α,L-xylo-2-hexulofuranose,
2,3-O-isopropylidene-1-deoxy-1-N-{[aminocarbonylaminophenylacetyl-α,L-Aspartyl-L-Valyl-L-Proline)}-α,L-xylo-2-hexulofuranose, and
2,3-O-isopropylidene-1 deoxy-1-N-{[aminocarbonylaminophenylacetyl-L-Valyl-L-Proline)}-α,L-xylo-2-hexulofuranose.

8. A method of inhibiting 5-lipoxygenase enzyme or related processes in a mammal comprising administering to the said animal a therapeutically effective amount of a compound selected from the group consisting of:
2,3-O-Isopropylidene-1-O-decyl-4-O-(methylaminocarbonyl)-6-deoxy-6-pyrrolidin-1-yl-α-L-xylo-2-hexulofuranose,
2,3-O-Isopropylidene-1-O-dodecyl-4-O-(methylaminocarbonyl)-6-deoxy-6-pyrrolidin-1-yl-α-L-xylo-2-hexulofuranose,
2,3-O-Isopropylidene-1-O-dodecyl-4-O-(methylaminocarbonyl)-6-deoxy-6-pyrrolidin-1-yl-α-L-xylo-2-hexulofuranose,
2,3-O-Isopropylidene-1-O-dodecyl-4-O-(4-chlorophenylaminocarbonyl)-6-deoxy-6-pyrrolidin-1-yl-α-L-xylo-2-hexulofuranose,
2,3-O-Isopropylidene-1-O-dodecyl-4-O-(4-tolylaminocarbonyl)-6-deoxy-6-pyrrolidin-1-yl-α-L-xylo-2-hexulofuranose,
2,3-O-Isopropylidene-1-O-dodecyl-4-O-(4-methoxyphenylaminocarbonyl)-6-deoxy-6-pyrrolidin-1-yl-α-L-xylo-2-hexulofuranose,
2,3-O-Isopropylidene-1-O-dodecyl-4-O-(4-nitrophenylaminocarbonyl)-6-deoxy-6-pyrrolidin-1-yl-α-L-xylo-2-hexulofuranose,
2,3-O-Isopropylidene-1-O-decyl-4-O-(phenylaminocarbonyl)-6-deoxy-6-pyrrolidin-1-yl-α-L-xylo-2-hexulofuranose,
2,3-O-Isopropylidene-1-O-decyl-4-O-(4-chlorophenylaminocarbonyl)-6-deoxy-6-pyrrolidin-1-yl-α-L-xylo-2-hexulofuranose,
2,3-O-Isopropylidene-1-O-decyl-4-O-(4-tolylaminocarbonyl)-6-deoxy-6-pyrrolidin-1-yl-α-L-xylo-2-hexulofuranose,
2,3-O-Isopropylidene-1-O-decyl-4-O-(4-methoxyphenylaminocarbonyl)-6-deoxy-6-pyrrolidin-1-yl-α-L-xylo-2-hexulofuranose,
2,3-O-Isopropylidene-1-O-decyl-4-O-(4-nitrophenylaminocarbonyl)-6-deoxy-6-pyrrolidin-1-yl-α-L-xylo-2-hexulofuranose,
2,3-O-Isopropylidene-1-O-heptyl-4-O-(phenylaminocarbonyl)-6-deoxy-6-pyrrolidin-1-yl-α-L-xylo-2-hexulofuranose,
2,3-O-Isopropylidene-1-O-heptyl-4-O-(4-chlorophenylaminocarbonyl)-6-deoxy-6-pyrrolidin-1-yl-α-L-xylo-2-hexulofuranose,
2,3-O-Isopropylidene-1-O-heptyl-4-O-(4-tolylaminocarbonyl)-6-deoxy-6-pyrrolidin-1-yl-α-L-xylo-2-hexulofuranose,
2,3-O-Isopropylidene-1-O-heptyl-4-O-(4-methoxyphenylaminocarbonyl)-6-deoxy-6-pyrrolidin-1-yl-α-L-xylo-2-hexulofuranose,
2,3-O-Isopropylidene-1-O-heptyl-4-O-(4-nitrophenylaminocarbonyl)-6-deoxy-6-pyrrolidin-1-yl-α-L-xylo-2-hexulofuranose,
2,3-O-Isopropylidene-1-O-dodecyl-4-O-(phenylaminocarbonyl)-6-deoxy-6-morpholin-4-yl-α-L-xylo-2-hexulofuranose,
2,3-O-Isopropylidene-1-O-dodecyl-4-O-(4-chlorophenylaminocarbonyl)-6-deoxy-6-morpholin-4-yl-α-L-xylo-2-hexulofuranose,
2,3-O-Isopropylidene-1-O-dodecyl-4-O-(4-tolylaminocarbonyl)-6-deoxy-6-morpholin-4-yl-α-L-xylo-2-hexulofuranose,
2,3-O-Isopropylidene-1-O-dodecyl-4-O-(4-methoxyphenylaminocarbonyl)-6-deoxy-6-morpholin-4-yl-α-L-xylo-2-hexulofuranose,
2,3-O-Isopropylidene-1-O-dodecyl-4-O-(4-nitrophenylaminocarbonyl)-6-deoxy-6-morpholin-4-yl-α-L-xylo-2-hexulofuranose,
2,3-O-Isopropylidene-1-O-decyl-4-O-(phenylaminocarbonyl)-6-deoxy-6-morpholin-4-yl-α-L-xylo-2-hexulofuranose,
2,3-O-Isopropylidene-1-O-decyl-4-O-(4-chlorophenylaminocarbonyl)-6-deoxy-6-morpholin-4-yl-α-L-xylo-2-hexulofuranose,
2,3-O-Isopropylidene-1-O-decyl-4-O-(4-tolylaminocarbonyl)-6-deoxy-6-morpholin-4-yl-α-L-xylo-2-hexulofuranose,
2,3-O-Isopropylidene-1-O-decyl-4-O-(4-methoxyphenylaminocarbonyl)-6-deoxy-6-morpholin-4-yl-α-L-xylo-2-hexulofuranose,
2,3-O-Isopropylidene-1-O-decyl-4-O-(4-nitrophenylaminocarbonyl)-6-deoxy-6-morpholin-4-yl-α-L-xylo-2-hexulofuranose,
2,3-O-Isopropylidene-1-O-heptyl-4-O-(phenylaminocarbonyl)-6-deoxy-6-morpholin-4-yl-α-L-xylo-2-hexulofuranose,
2,3-O-Isopropylidene-1-O-heptyl-4-O-(4-chlorophenylaminocarbonyl)-6-deoxy-6-morpholin-4-yl-α-L-xylo-2-hexulofuranose,
2,3-O-Isopropylidene-1-O-heptyl-4-O-(4-tolylaminocarbonyl)-6-deoxy-6-morpholin-4-yl-α-L-xylo-2-hexulofuranose,
2,3-O-Isopropylidene-1-O-heptyl-4-O-(4-methoxyphenylaminocarbonyl)-6-deoxy-6-morpholin-4-yl-α-L-xylo-2-hexulofuranose,
2,3-O-Isopropylidene-1-O-heptyl-4-O-(4-nitrophenylaminocarbonyl)-6-deoxy-6-morpholin-4-yl-α-L-xylo-2-hexulofuranose,
2,3-O-Isopropylidene-1-O-dodecyl-4-O-(phenylaminocarbonyl)-6-deoxy-6-piperidin-1-yl-α-L-xylo-2-hexulofuranose,
2,3-O-Isopropylidene-1-O-dodecyl-4-O-(4-chlorophenylaminocarbonyl)-6-deoxy-6-piperidin-1-yl-α-L-xylo-2-hexulofuranose,
2,3-O-Isopropylidene-1-O-dodecyl-4-O-(4-tolylaminocarbonyl)-6-deoxy-6-piperidin-1-yl-α-L-xylo-2-hexulofuranose,
2,3-O-Isopropylidene-1-O-dodecyl-4-O-(4-methoxyphenylaminocarbonyl)-6-deoxy-6-piperidin-1-yl-α-L-xylo-2-hexulofuranose,
2,3-O-Isopropylidene-1-O-dodecyl-4-O-(4-nitrophenylaminocarbonyl)-6-deoxy-6-piperidin-1-yl-α-L-xylo-2-hexulofuranose,
2,3-O-Isopropylidene-1-O-decyl-4-O-(phenylaminocarbonyl)-6-deoxy-6-piperidin-1-yl-α-L-xylo-2-hexulofuranose,
2,3-O-Isopropylidene-1-O-decyl-4-O-(4-chlorophenylaminocarbonyl)-6-deoxy-6-piperidin-1-yl-α-L-xylo-2-hexulofuranose,
2,3-O-Isopropylidene-1-O-decyl-4-O-(4-tolylaminocarbonyl)-6-deoxy-6-piperidin-1-yl-α-L-xylo-2-hexulofuranose,
2,3-O-Isopropylidene-1-O-decyl-4-O-(4-methoxyphenylaminocarbonyl)-6-deoxy-6-piperidin-1-yl-α-L-xylo-2-hexulofuranose,
2,3-O-Isopropylidene-1-O-decyl-4-O-(4-nitrophenylaminocarbonyl)-6-deoxy-6-piperidin-1-yl-α-L-xylo-2-hexulofuranose,
2,3-O-Isopropylidene-1-O-heptyl-4-O-(phenylaminocarbonyl)-6-deoxy-6-piperidin-1-yl-α-L-xylo-2-hexulofuranose,
2,3-O-Isopropylidene-1-O-heptyl-4-O-(4-chlorophenylaminocarbonyl)-6-deoxy-6-piperidin-1-yl-α-L-xylo-2-hexulofuranose,
2,3-O-Isopropylidene-1-O-heptyl-4-O-(4-tolylaminocarbonyl)-6-deoxy-6-piperidin-1-yl-α-L-xylo-2-hexulofuranose,
2,3-O-Isopropylidene-1-O-heptyl-4-O-(4-methoxyphenylaminocarbonyl)-6-deoxy-6-piperidin-1-yl-α-L-xylo-2-hexulofuranose,
2,3-O-Isopropylidene-1-O-heptyl-4-O-(4-nitrophenylaminocarbonyl)-6-deoxy-6-piperidin-1-yl-α-L-xylo-2-hexulofuranose,
2,3-O-Isopropylidene-1-O-dodecyl-4-O-(phenylaminocarbonyl)-6-deoxy-6-azepan-1-yl-α-L-xylo-2-hexulofuranose,
2,3-O-Isopropylidene-1-O-dodecyl-4-O-(4-chlorophenylaminocarbonyl)-6-deoxy-6-azepan-1-yl-α-L-xylo-2-hexulofuranose,
2,3-O-Isopropylidene-1-O-dodecyl-4-O-(4-tolylaminocarbonyl)-6-deoxy-6-azepan-1-yl-α-L-xylo-2-hexulofuranose,
2,3-O-Isopropylidene-1-O-dodecyl-4-O-(4-methoxyphenylaminocarbonyl)-6-deoxy-6-azepan-1-yl-α-L-xylo-2-hexulofuranose,
2,3-O-Isopropylidene-1-O-dodecyl-4-O-(4-nitrophenylaminocarbonyl)-6-deoxy-6-azepan-1-yl-α-L-xylo-2-hexulofuranose,
2,3-O-Isopropylidene-1-O-decyl-4-O-(phenylaminocarbonyl)-6-deoxy-6-azepan-1-yl-α-L-xylo-2-hexulofuranose,
2,3-O-Isopropylidene-1-O-decyl-4-O-(4-chlorophenylaminocarbonyl)-6-deoxy-6-azepan-1-yl-α-L-xylo-2-hexulofuranose,
2,3-O-Isopropylidene-1-O-decyl-4-O-(4-tolylaminocarbonyl)-6-deoxy-6-azepan-1-yl-α-L-xylo-2-hexulofuranose,
2,3-O-Isopropylidene-1-O-decyl-4-O-(4-methoxyphenylaminocarbonyl)-6-deoxy-6-azepan-1-yl-α-L-xylo-2-hexulofuranose,
2,3-O-Isopropylidene-1-O-decyl-4-O-(4-nitrophenylaminocarbonyl)-6-deoxy-6-azepan-1-yl-α-L-xylo-2-hexulofuranose,
2,3-O-Isopropylidene-1-O-heptyl-4-O-(phenylaminocarbonyl)-6-deoxy-6-azepan-1-yl-α-L-xylo-2-hexulofuranose,
Hydrochloride salt of 2,3-O-Isopropylidene-1-O-heptyl-4-O-(phenylaminocarbonyl)-6-deoxy-6-azepan-1-yl-α-L-xylo-2-hexulofuranose,
2,3-O-Isopropylidene-1-O-heptyl-4-O-(4-chlorophenylaminocarbonyl)-6-deoxy-6-azepan-1-yl-α-L-xylo-2-hexulofuranose,
2,3-O-Isopropylidene-1-O-heptyl-4-O-(4-tolylaminocarbonyl)-6-deoxy-6-azepan-1-yl-α-L-xylo-2-hexulofuranose,
Hydrochloride salt of 2,3-O-Isopropylidene-1-O-heptyl-4-O-(4-tolylaminocarbonyl)-6-deoxy-6-azepan-1-yl-α-L-xylo-2-hexulofuranose,
2,3-O-Isopropylidene-1-O-heptyl-4-O-(4-methoxyphenylaminocarbonyl)-6-deoxy-6-azepan-1-yl-α-L-xylo-2-hexulofuranose,
2,3-O-Isopropylidene-1-O-heptyl-4-O-(4-nitrophenylaminocarbonyl)-6-deoxy-6-azepan-1-yl-α-L-xylo-2-hexulofuranose,
2,3-O-Isopropylidene-1-O-dodecyl-4-O-(phenylaminocarbonyl)-6-deoxy-6-[2-(pyrrolidin-1-yl)ethyl]amino-α-L-xylo-2-hexulofuranose,
2,3-O-Isopropylidene-1-O-dodecyl-4-O-(4-chlorophenylaminocarbonyl)-6-deoxy-6-[2-(pyrrolidin-1-yl)ethyl]amino-α-L-xylo-2-hexulofuranose,
2,3-O-Isopropylidene-1-O-dodecyl-4-O-(4-tolylaminocarbonyl)-6-deoxy-6-[2-(pyrrolidin-1-yl)ethyl]amino-α-L-xylo-2-hexulofuranose,
2,3-O-Isopropylidene-1-O-dodecyl-4-O-(4-methoxyphenylaminocarbonyl)-6-deoxy-6-[2-(pyrrolidin-1-yl)ethyl]amino-α-L-xylo-2-hexulofuranose,
2,3-O-Isopropylidene-1-O-dodecyl-4-O-(4-nitrophenylaminocarbonyl)-6-deoxy-6-[2-(pyrrolidin-1-yl)ethyl]amino-α-L-xylo-2-hexulofuranose,
2,3-O-Isopropylidene-1-O-decyl-4-O-(phenylaminocarbonyl)-6-deoxy-6-[2-(pyrrolidin-1-yl)ethyl]amino-α-L-xylo-2-hexulofuranose,
2,3-O-Isopropylidene-1-O-decyl-4-O-(4-chlorophenylaminocarbonyl)-6-deoxy-6-[2-(pyrrolidin-1-yl)ethyl]amino-α-L-xylo-2-hexulofuranose,
2,3-O-Isopropylidene-1-O-decyl-4-O-(4-tolylaminocarbonyl)-6-deoxy-6-[2-(pyrrolidin-1-yl)ethyl]amino-α-L-xylo-2-hexulofuranose,
2,3-O-Isopropylidene-1-O-decyl-4-O-(4-methoxyphenylaminocarbonyl)-6-deoxy-6-[2-(pyrrolidin-1-yl)ethyl]amino-α-L-xylo-2-hexulofuranose,
2,3-O-Isopropylidene-1-O-decyl-4-O-(4-nitrophenylaminocarbonyl)-6-deoxy-6-[2-(pyrrolidin-1-yl)ethyl]amino-α-L-xylo-2-hexulofuranose,
2,3-O-Isopropylidene-1-O-heptyl-4-O-(phenylaminocarbonyl)-6-deoxy-6-[2-(pyrrolidin-1-yl)ethyl]amino-α-L-xylo-2-hexulofuranose,
2,3-O-Isopropylidene-1-O-heptyl-4-O-(4-chlorophenylaminocarbonyl)-6-deoxy-6-[2-(pyrrolidin-1-yl)ethyl]amino-α-L-xylo-2-hexulofuranose,
2,3-O-Isopropylidene-1-O-heptyl-4-O-(4-tolylaminocarbonyl)-6-deoxy-6-[2-(pyrrolidin-1-yl)ethyl]amino-α-L-xylo-2-hexulofuranose,
2,3-O-Isopropylidene-1-O-heptyl-4-O-(4-methoxyphenylaminocarbonyl)-6-deoxy-6-[2-(pyrrolidin-1-yl)ethyl]amino-α-L-xylo-2-hexulofuranose,
2,3-O-Isopropylidene-1-O-heptyl-4-O-(4-nitrophenylaminocarbonyl)-6-deoxy-6-[2-(pyrrolidin-1-yl)ethyl]amino-α-L-xylo-2-hexulofuranose,
2,3-O-Isopropylidene-1-O-dodecyl-4-O-(phenylaminocarbonyl)-6-deoxy-6-[2-(piperidin-1-yl)ethyl]amino-α-L-xylo-2-hexulofuranose,
2,3-O-Isopropylidene-1-O-dodecyl-4-O-(4-chlorophenylaminocarbonyl)-6-deoxy-6-[2-(piperidin-1-yl)ethyl]amino-α-L-xylo-2-hexulofuranose,
2,3-O-Isopropylidene-1-O-dodecyl-4-O-(4-tolylaminocarbonyl)-6-deoxy-6-[2-(piperidin-1-yl)ethyl]amino-α-L-xylo-2-hexulofuranose,
2,3-O-Isopropylidene-1-O-dodecyl-4-O-(4-methoxyphenylaminocarbonyl)-6-deoxy-6-[2-(piperidin-1-yl)ethyl]amino-α-L-xylo-2-hexulofuranose,
2,3-O-Isopropylidene-1-O-dodecyl-4-O-(4-nitrophenylaminocarbonyl)-6-deoxy-6-[2-(piperidin-1-yl)ethyl]amino-α-L-xylo-2-hexulofuranose,
2,3-O-Isopropylidene-1-O-decyl-4-O-(phenylaminocarbonyl)-6-deoxy-6-[2-(piperidin-1-yl)ethyl]amino-α-L-xylo-2-hexulofuranose,
2,3-O-Isopropylidene-1-O-decyl-4-O-(4-chlorophenylaminocarbonyl)-6-deoxy-6-[2-(piperidin-1-yl)ethyl]amino-α-L-xylo-2-hexulofuranose,
2,3-O-Isopropylidene-1-O-decyl-4-O-(4-tolylaminocarbonyl)-6-deoxy-6-[2-(piperidin-1-yl)ethyl]amino-α-L-xylo-2-hexulofuranose,
2,3-O-Isopropylidene-1-O-decyl-4-O-(4-methoxyphenylaminocarbonyl)-6-deoxy-6-[2-(piperidin-1-yl)ethyl]amino-α-L-xylo-2-hexulofuranose,
2,3-O-Isopropylidene-1-O-decyl-4-O-(4-nitrophenylaminocarbonyl)-6-deoxy-6-[2-(piperidin-1-yl)ethyl]amino-α-L-xylo-2-hexulofuranose,
2,3-O-Isopropylidene-1-O-heptyl-4-O-(phenylaminocarbonyl)-6-deoxy-6-[2-(piperidin-1-yl)ethyl]amino-α-L-xylo-2-hexulofuranose,
2,3-O-Isopropylidene-1-O-heptyl-4-O-(4-chlorophenylaminocarbonyl)-6-deoxy-6-[2-(piperidin-1-yl)ethyl]amino-α-L-xylo-2-hexulofuranose,
2,3-O-Isopropylidene-1-O-heptyl-4-O-(4-tolylaminocarbonyl)-6-deoxy-6-[2-(piperidin-1-yl)ethyl]amino-α-L-xylo-2-hexulofuranose,
2,3-O-Isopropylidene-1-O-heptyl-4-O-(4-methoxyphenylaminocarbonyl)-6-deoxy-6-[2-(piperidin-1-yl)ethyl]amino-α-L-xylo-2-hexulofuranose,
2,3-O-Isopropylidene-1-O-heptyl-4-O-(4-nitrophenylaminocarbonyl)-6-deoxy-6-[2-(piperidin-1-yl)ethyl]amino-α-L-xylo-2-hexulofuranose,
2,3-O-Isopropylidene-1-O-dodecyl-4-O-(phenylaminocarbonyl)-6-deoxy-6-[2-(morphilin-4-yl)ethyl]amino-α-L-xylo-2-hexulofuranose,
2,3-O-Isopropylidene-1-O-dodecyl-4-O-(4-chlorophenylaminocarbonyl)-6-deoxy-6-(2-ethylmorphilin-4-yl)-α-L-xylo-2-hexulofuranose,
2,3-O-Isopropylidene-1-O-dodecyl-4-O-(4-tolylaminocarbonyl)-6-deoxy-6-[2-(morphilin-4-yl)ethyl]amino-α-L-xylo-2-hexulofuranose,
2,3-O-Isopropylidene-1-O-dodecyl-4-O-(4-methoxyphenylaminocarbonyl)-6-deoxy-6-[2-(morphilin-4-yl)ethyl]amino-α-L-xylo-2-hexulofuranose,
2,3-O-Isopropylidene-1-O-dodecyl-4-O-(4-nitrophenylaminocarbonyl)-6-deoxy-6-[2-(morphilin-4-yl)ethyl]amino-α-L-xylo-2-hexulofuranose,
2,3-O-Isopropylidene-1-O-decyl-4-O-(phenylaminocarbonyl)-6-deoxy-6-[2-(morphilin-4-yl)ethyl]amino-α-L-xylo-2-hexulofuranose,
2,3-O-Isopropylidene-1-O-decyl-4-O-(4-chlorophenylaminocarbonyl)-6-deoxy-6-[2-(morphilin-4-yl)ethyl]amino-α-L-xylo-2-hexulofuranose,
2,3-O-Isopropylidene-1-O-decyl-4-O-(4-tolylaminocarbonyl)-6-deoxy-6-[2-(morphilin-4-yl)ethyl]amino-α-L-xylo-2-hexulofuranose,
Hydrochloride salt of 2,3-O-Isopropylidene-1-O-decyl-4-O-(4-tolylaminocarbonyl)-6-deoxy-6-[2-(morphilin-4-yl)ethyl]amino-α-L-xylo-2-hexulofuranose,
2,3-O-Isopropylidene-1-O-decyl-4-O-(4-methoxyphenylaminocarbonyl)-6-deoxy-6-[2-(morphilin-4-yl)ethyl]amino-α-L-xylo-2-hexulofuranose,
2,3-O-Isopropylidene-1-O-dodecyl-4-O-(4-nitrophenylaminocarbonyl)-6-deoxy-6-[2-(morphilin-4-yl)ethyl]amino-α-L-xylo-2-hexulofuranose,
2,3-O-Isopropylidene-1-O-heptyl-4-O-(phenylaminocarbonyl)-6-deoxy-6-[2-(morphilin-4-yl)ethyl]amino-α-L-xylo-2-hexulofuranose,
2,3-O-Isopropylidene-1-O-heptyl-4-O-(4-chlorophenylaminocarbonyl)-6-deoxy-6-[2-(morphilin-4-yl)ethyl]amino-α-L-xylo-2-hexulofuranose,
2,3-O-Isopropylidene-1-O-heptyl-4-O-(4-tolylaminocarbonyl)-6-deoxy-6-[2-(morphilin-4-yl)ethyl]amino-α-L-xylo-2-hexulofuranose,
2,3-O-Isopropylidene-1-O-heptyl-4-O-(4-methoxyphenylaminocarbonyl)-6-deoxy-6-[2-(morphilin-4-yl)ethyl]amino-α-L-xylo-2-hexulofuranose,
2,3-O-Isopropylidene-1-O-heptyl-4-O-(4-nitrophenylaminocarbonyl)-6-deoxy-6-[2-(morphilin-4-yl)ethyl]amino-α-L-xylo-2-hexulofuranose,
2,3,O-Isopropylidene-1-O-dodecyl-4-O-(methylaminocarbonyl)-6-deoxy-6-azepan-1-yl-α-L-xylo-2-hexulofuranose,
2,3,O-Isopropylidene-1-O-dodecyl-4-O-(methylaminocarbonyl)-6-deoxy-6-morpholin-4-yl-α-L-xylo-2-hexulofuranose,
2,3,O-Isopropylidene-1-O-dodecyl-4-O-(methylaminocarbonyl)-6-deoxy-6-piperidin-1-yl-α-L-xylo-2-hexulofuranose,
2,3,O-Isopropylidene-1-O-dodecyl-4-O-(methylaminocarbonyl)-6-deoxy-6-[2-(pyrrolidin-1-yl)ethyl]amino-α-L-xylo-2-hexulofuranose,
2,3,O-Isopropylidene-1-O-dodecyl-4-O-(methylaminocarbonyl)-6-deoxy-6-[2-(morphilin-4-yl)ethyl]amino-α-L-xylo-2-hexulofuranose,
2,3,0-Isopropylidene-1-O-decyl-4-O-(methylaminocarbonyl)-6-deoxy-6-morpholin-4-yl-α-L-xylo-2-hexulofuranose,
2,3,O-Isopropylidene-1-O-decyl-4-O-(methylaminocarbonyl)-6-deoxy-6-azepan-1-yl-α-L-xylo-2-hexulofuranose,
2,3,O-Isopropylidene-1-O-decyl-4-O-(methylaminocarbonyl)-6-deoxy-6-piperidin-1-yl-α-L-xylo-2-hexulofuranose,
2,3,O-Isopropylidene-1-O-decyl-4-O-(methylaminocarbonyl)-6-deoxy-6-[2-(pyrrolidin-1-yl)ethyl]amino-α-L-xylo-2-hexulofuranose,
2,3,O-Isopropylidene-1-O-decyl-4-O-(methylaminocarbonyl)-6-deoxy-6-[2-(morphilin-4-yl)ethyl]amino-α-L-xylo-2-hexulofuranose,
2,3,O-Isopropylidene-1-O-heptyl-4-O-(methylaminocarbonyl)-6-deoxy-6-pyrrolidin-1-yl-α-L-xylo-2-hexulofuranose,
2,3,O-Isopropylidene-1-O-heptyl-4-O-(methylaminocarbonyl)-6-deoxy-6-morpholin-4-yl-α-L-xylo-2-hexulofuranose,
2,3,O-Isopropylidene-1-O-heptyl-4-O-(methylaminocarbonyl)-6-deoxy-6-azepan-1-yl-α-L-xylo-2-hexulofuranose,
2,3,O-Isopropylidene-1-O-heptyl-4-O-(methylaminocarbonyl)-6-deoxy-6-piperidin-1-yl-α-L-xylo-2-hexulofuranose,
2,3,O-Isopropylidene-1-O-heptyl-4-O-(methylaminocarbonyl)-6-deoxy-6-[2-(pyrrolidin-1-yl)ethyl]amino-α-L-xylo-2-hexulofuranose, and
2,3,O-Isopropylidene-1-O-heptyl-4-O-(methylaminocarbonyl)-6-deoxy-6-[2-(morphilin-4-yl)ethyl]amino-α-L-xylo-2-hexulofuranose.

9. A method of inhibiting 5-lipoxygenase enzyme or related processes in a mammal comprising administering to the animal a therapeutically effective amount of a pharmaceutical composition of a compound as claimed in any of the claims 1-4 or a pharmaceutically acceptable salt thereof and a pharmaceutically acceptable carrier.

10. A method of inhibiting, preventing or suppressing the generation of leukotrienes, and related substances comprising administering to the mammal a therapeutically effective amount of a compound as claimed in any of the claims 1-4 or a pharmaceutically acceptable salt thereof and a pharmaceutically acceptable carrier.

11. The method according to claim 10, wherein the related substances are selected from the group comprising of cytokines, chemokine gene expressions and proteolytic enzymes.

12. A therapeutic method comprising treatment of a pathology wherein 5-lipoxygenase activation is implicated and inhibition of 5-lipoxygenase is accomplished by administering a compound claimed in any of the claims 1-4 to a mammal in need of such therapy.

13. The method according to claim 12, wherein the treatment comprises the prophylaxis or direct treatment of a mammal suffering from allergic rhinitis.

14. The method according to claim 12, wherein the treatment comprising the prophylaxis or direct treatment of an animal suffering from chronic obstructive pulmonary disease (COPD).
